(19) United States
(12) Patent Application Publication
(10) Pub. No.: US 2018/0243190 A1
(43) Pub. Date: Aug. 30, 2018

(54) ACTIVATORS OF NRF2-DEPENDENT PHOTOPROTECTION AND RELATED USES THEREOF

(71) Applicant: THE ARIZONA BOARD OF REGENTS ON BEHALF OF THE UNIVERSITY OF ARIZONA, Tucson, AZ (US)

(72) Inventors: Georg T. WONDRAK, Tucson, AZ (US); Donna D. ZHANG, Tucson, AZ (US)

(21) Appl. No.: 15/753,514

(22) PCT Filed: Aug. 17, 2016
(86) PCT No.: PCT/US2016/047390

§ 371 Date: Feb. 19, 2018

Related U.S. Application Data
(60) Provisional application No. 62/206,548, filed on Aug. 18, 2015.

(51) Int. Cl. A61K 8/37; A61Q 17/04; A61K 8/86

(52) U.S. Cl. CPC A61K 8/37; A61K 8/86; A61Q 17/04

(57) ABSTRACT

Provided herein are methods for preventing conditions related to UV-radiation exposure in subjects at risk for exposure to UV-radiation. In particular, the invention relates to compositions comprising specific formulations of dietary carotenoids (e.g., bixin) which function as activators of NRF2 pathway related activity, and related methods for the protection of mammalian skin against UV-radiation.

### CROSS-REFERENCE TO RELATED APPLICATIONS

This application claims priority to and the benefit of U.S. Provisional Application No. 62/206,548, filed Aug. 18, 2015, which is hereby incorporated by reference in its entirety.

### FIELD OF THE INVENTION

Provided herein are methods for preventing conditions related to UV-radiation and exposure to other photons (e.g. visible and ionizing radiation) in subjects at risk for photon exposure including from UV-radiation. In particular, the invention relates to compositions comprising specific formulations of dietary carotenoids (e.g., bixin) which function as activators of NRF2 pathway related activity, and related methods for the protection of mammalian skin against UV-radiation and other types of high energy photons (e.g. visible and ionizing radiation).

### INTRODUCTION

According to the U.S. Department of Health and Human Services and the World Health Organization, ultraviolet (UV) radiation, from the sun and from tanning beds, is classified as a human carcinogen. Scientists classify UV radiation generally into three types or bands, i.e., UVA, UVB and UVC. Even though the stratospheric ozone layer absorbs some of the harmful UV emitted from the sun, it does not screen all UV radiation. For example, while UVA, which is emitted at wavelength 320-400 nm, is not absorbed by the ozone layer, UVB, which is emitted at wavelength 290-320 nm, is mostly absorbed by the ozone layer, but some nevertheless does reach the Earth's surface. UVC, which is emitted at wavelength 100-290 nm, is generally believed to be completely absorbed by the ozone layer and atmosphere. UVA and UVB radiation that reaches the Earth's surface contributes to the serious health effects listed above; it also contributes to environmental impacts. Levels of UVA radiation are more constant than UVB, reaching the Earth's surface without variations due to the time of day or year. UVA radiation is not filtered by glass.

The sun emits energy over a broad spectrum of wavelengths: visible light that you see, infrared radiation that you feel as heat, and UV radiation that you can't see or feel. UV radiation has a shorter wavelength and higher energy than visible light. It affects human health both positively and negatively. Short exposure to UVB radiation generates vitamin D, but can also lead to sunburn depending on an individual's skin type. As indicated above, while the stratospheric ozone layer shields life on Earth from most UV radiation, what does get through the ozone layer can cause numerous health problems, particularly for people who spend unprotected time outdoors or who are at greater risk to UV exposure. Such problems include skin cancer, cataracts, suppression of the immune system and premature aging of the skin.

Because the benefits of sunlight cannot be separated from its damaging effects, it is important to understand the risks of overexposure. Sunlight causes photodamage to skin which in turn causes it to age faster than it should. Thus, skin age and a person's age may not necessarily be the same. Photodamaged or sun-damaged skin is something that few people escape in their lifetime. Photodamage results from exposure to sunlight or other sources of UV such as tanning beds, whether or not sun-tanning is involved. Approximately twenty five percent of lifetime UV exposure generally happens before people reach the age of twenty. UV-damaged or photodamaged skin manifests in numerous ways, such as advanced aging or wrinkling, thickening of the skin, i.e., the leathery, weather-beaten, elephant hide look (skin will generally thicken all over when people sun bake), uneven or pebbly skin, flabbiness, lifeless skin, pigmentation irregularities, small dilated blood vessels or red markings on or near the surface of the skin also known as telangiectasias, rough or scaly patches, e.g., actinic keratoses, freckles otherwise known as ephilides, liver spots, age spots, dark spots or skin tags known as lentigines, pre-skin cancers, and skin cancer, such as non-melanoma skin cancer (NMSC), e.g., superficial basal cell carcinoma (sBCC) and squamous cell carcinoma (SCC), and malignant melanoma.

Generally, these changes occur more frequently on areas that experience chronic exposure, such as the face, head, neck, chest, ears, arms, hands, backs and legs. Because the buttocks and upper inner arms are often unexposed, these areas of skin generally remain pristine evidencing the difference between chronologic aging and photoaging.

As the manifestations of photodamage intensify with age, it is paramount to seek medical advice and treatment, preferably early on, to mitigate and even possibly reverse some of the effects of photodamage to skin.

As such, improved methods for protecting photodamage to the skin are needed.

### SUMMARY OF THE INVENTION

Exposure to solar ultraviolet (UV) radiation is a causative factor in skin photodamage and carcinogenesis, and an urgent need exists for improved molecular photoprotective strategies different from (or synergistic with) photon absorption. Recent studies suggest a photoprotective role of cutaneous gene expression orchestrated by the transcription factor NRF2 (nuclear factor-E2-related factor 2).

Experiments conducted during the course of developing embodiments explored the molecular mechanism underlying carotenoid-based systemic skin photoprotection in SKH-1 mice and provide genetic evidence that photoprotection achieved by the FDA-approved apocarotenoid and food additive bixin depends on NRF2 activation. It was shown that bixin activates NRF2 through the critical Cys-151 sensor residue in KEAP1, orchestrating a broad cytoprotective response in cultured human keratinocytes as revealed by antioxidant gene expression array analysis. Following dose optimization studies for cutaneous NRF2 activation by systemic administration of bixin, feasibility of bixin-based suppression of acute cutaneous photodamage from solar UV exposure was investigated in Nrf2^{+/+} versus Nrf2^{-/-} SKH-1 mice. Systemic administration of bixin suppressed skin photodamage, attenuating epidermal oxidative DNA damage and inflammatory responses in Nrf2^{+/+} but not in Nrf2^{-/-} mice, confirming the NRF2-dependence of bixin-based cytoprotection. It was further demonstrated that administration of 1% bixin in PEG based carrier activates Nrf2 and Nrf2 target expression in skin tissues of SKH-1 mice, but not in a standard topical carrier (e.g., Vanicream). It was further demonstrated that bixin treatment induces Nrf2 and Nrf2 target gene expression in human primary skin melanocytes. It was further demonstrated that irradiation of bixin with solar ultraviolet light enhances (potentiates′) bixin activity for upregulation of cytoprotective gene expression in human skin keratinocytes.

Taken together, these data indicate feasibility of achieving NRF2-dependent cutaneous photoprotection by systemic administration of the apocarotenoid bixin, a natural food additive consumed worldwide.

Accordingly, provided herein are methods for preventing conditions related to UV-radiation exposure and exposure to other photons (e.g. visible and ionizing radiation) in subjects at risk for such exposure. In particular, the invention relates to compositions comprising specific formulations of dietary carotenoids (e.g., bixin) which function as activators of NRF2 pathway related activity, and related methods for the protection of mammalian skin against UV-radiation. In some embodiments, such methods are also useful for preventing conditions related to photons in the non-UV range.

In certain embodiments, the present invention provides a new and improved prophylactic prevention of UV-related skin damage in a subject (e.g., a human subject at risk for exposure to UV-radiation) with an effective amount of a composition comprising the apocarotenoid bixin, regardless of the UV-type, e.g., UV-A, UV-B or UV-C.

Such methods require that the amount of bixin delivered to the subject be sufficient to activate the NRF2 pathway related activity (e.g., through engagement with the Cys151 residue of the KEAP1 protein) within skin cells at risk for exposure to UV-radiation. In some embodiments, the dosage amount of bixin is between approximately 10 mg/kg (e.g., 5, 6, 7, 8, 9, 10, 11, 12, 13, 14, 15, 20, 30 mg/kg) and 200 mg/kg (e.g., 175, 180, 190, 200, 201, 205, 220, 300 mg/kg) of the subject. Indeed, experiments conducted during the course of developing embodiments for the present invention determined that dosage amounts less than 10 mg/kg of the subject were insufficient to activate the NRF2 pathway related activity (e.g., through engagement with the Cys151 residue of the KEAP1 protein) within skin cells at risk for exposure to UV-radiation.

As noted, experiments conducted during the course of developing embodiments for the present invention demonstrated that irradiation of bixin with solar ultraviolet light enhances (potentiates′) bixin activity for upregulation of cytoprotective gene expression in human skin keratinocytes. As such, in some embodiments, the bixin is irradiated bixin. In some embodiments, the source of irradiation is selected from ultraviolet light, visible light, and ionizing radiation. In some embodiments, the result of irradiation is the formation of photochemical bixin derivatives and degradation products.

Such methods are not limited to a particular manner of administering the composition comprising bixin.

In some embodiments, the composition comprising bixin is administered topically in a skin area at risk for exposure to UV-radiation (e.g., in the form of a cream, gel, oil, or lotion). In some embodiments involving topical administration, the bixin is within a composition further comprising polyethylene glycol. In such embodiments, the amount of bixin within a composition comprising bixin and polyethylene glycol is approximately 1% (e.g., 0.5%, 0.7%, 0.85%, 0.9%, 0.95%, 0.999%, 1%, 1.05%, 1.1%, 1.5%, 1.75%, 2%, 2.5%, etc.).

In some embodiments, the composition comprising bixin is orally administered to achieve systemic administration. Indeed, the manner of administration is irrelevant so long as the resulting administration results in activation of NRF2 pathway related activity (e.g., through engagement with the Cys151 residue of the KEAP1 protein) within skin cells at risk for exposure to UV-radiation. In some embodiments, the administration results in activation of NRF2 pathway related activity in skin cells including, but not limited to, keratinocyte cells and/or pigment cells (e.g., melanocyte cells).

In certain embodiments, the present invention provides a new and improved prophylactic prevention of skin damage in a subject related to photons outside of the UV range with an effective amount of a composition comprising the apocarotenoid bixin.

In certain embodiments, the present invention provides a new and improved prophylactic prevention of skin damage in a subject related to environmental stressors (e.g., electrophilic environmental stressors) with an effective amount of a composition comprising the apocarotenoid bixin. Examples of such environmental stressors include, but are not limited to, pollutants such as diesel exhaust, benzpyrene, dioxin; metals and metalloids:arsenic, cadmium; gases/smog: ozone; nitrogen oxides; halogen-based pool disinfectants, solar UV, ionizing radiation; visible light, infrared; radioactivity: Radon etc.

In certain embodiments of the invention, combination prophylactic treatment of animals at risk for UV-radiation skin damage with a therapeutically effective amount of a composition comprising bixin and a course of an additional photoprotective agent known to prevent UV-radiation related skin damage produces a greater prevention of UV-radiation related skin damage and clinical benefit in such animals compared to those treated with agent known to prevent UV-radiation related skin damage (e.g., the additional photoprotective agent alone).

In some embodiments, the bixin is irradiated bixin. In some embodiments, the source of irradiation is selected from ultraviolet light, visible light, and ionizing radiation. In some embodiments, the result of irradiation is the formation of photochemical bixin derivatives and degradation products.

In some embodiments, the bixin is within a composition further comprising polyethylene glycol. In such embodiments, the amount of bixin within a composition comprising bixin and polyethylene glycol is approximately 1% (e.g., 0.5%, 0.7%, 0.85%, 0.9%, 0.95%, 0.999%, 1%, 1.05%, 1.1%, 1.5%, 1.75%, 2%, 2.5%, etc.). In some embodiments, the composition comprising bixin is a part of a larger composition known to prevent UV-radiation related skin damage (e.g., a part of the additional photoprotective agent). Examples of additional protective agents include, but are not limited to, sun screen, sunblock, suntan lotion, sunburn cream, sun cream and block out. In some embodiments, the additional photoprotective agent is a composition comprising effective amounts of titanium dioxide. In some embodiments, the additional photoprotective agent is a composition comprising effective amounts of zinc oxide. In some embodiments, the additional photoprotective agent is a composition comprising effective amounts of titanium dioxide and zinc oxide. In some embodiments, the additional photoprotective agent is a composition comprising effective amounts of one or more of the following: p-aminobenzoic acid, padimate 0, phenylbenzimidazole sulfonic acid, cinoxate, dioxybenzone, oxybenzone, homosalate, menthyl anthranilate, octocrylene, octyl methoxycinnamate, octyl salicylate, sulisobenzone, trolamine salicylate, avobenzone, ecamsule, titanium dioxide, zinc oxide, 4-methylbenzylidene camphor, tinosorb M, tinosorb S, tinosorb A2B, neo heliopan AP, mexoryl XL, benzophenone-9, uvinul T 150, uvinul A Plus, uvasorb HEB, parsol SLX, and amiloxate.

As noted, the Applicants have found that effective amounts of the apocarotenoid bixin

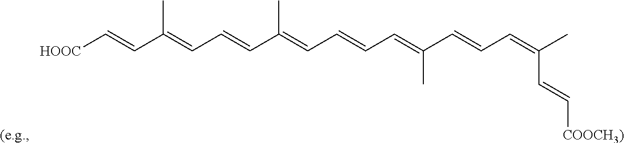

functions as an activator of NRF2 pathway related activity (e.g., through engagement with the Cys151 residue of the KEAP1 protein) within skin cells (e.g., keratinocytes, melanocytes) at risk for exposure to UV-radiation.

The invention also provides pharmaceutical compositions comprising the compounds of the invention in a pharmaceutically acceptable carrier. As such, in certain embodiments, the present invention provides pharmaceutical compositions comprising effective amounts of the apocarotenoid bixin, or variants thereof, wherein the composition has cutaneous photoprotective properties against A or B forms of ultraviolet radiation. In some embodiments, the pharmaceutical composition functions as an activator of NRF2 pathway related activity (e.g., through engagement with the Cys151 residue of the KEAP1 protein) within skin cells at risk for exposure to UV-radiation. In some embodiments, the skin cells include keratinocyte cells and/or pigment cells (e.g., melanocyte cells). In some embodiments, effective amounts of compounds structurally similar to bixin (e.g., norbixin) which also are capable of activating NRF2 pathway related activity (e.g., through engagement with the Cys151 residue of the KEAP1 protein) within skin cells at risk for exposure to UV-radiation are provided. In some embodiments, any compound capable of activating NRF2 pathway related activity (e.g., through engagement with the Cys151 residue of the KEAP1 protein) within skin cells at risk for exposure to UV-radiation is provided. Indeed, such compounds may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

The invention further provides processes for preparing any of the compounds of the present invention.

The invention also provides the use of compounds capable of activating NRF2 pathway related activity (e.g., through engagement with the Cys151 residue of the KEAP1 protein) within skin cells at risk for exposure to UV-radiation (e.g., bixin and compound structurally similar to bixin) (e.g., irradiated bixin) for purposes of protecting skin from conditions caused by UV-radiation exposure.

Examples of conditions caused by UV-radiation exposure include, but are not limited to, advanced skin aging or wrinkling, thickening of the skin (e.g., the leathery, weather-beaten, elephant hide look), uneven or pebbly skin, flabbiness, lifeless skin, pigmentation irregularities, small dilated blood vessels or red markings on or near the surface of the skin also known as telangiectasias, rough or scaly patches, e.g., actinic keratoses, freckles otherwise known as ephilides, liver spots, age spots, dark spots or skin tags known as lentigines, pre-skin cancers, and skin cancer, such as non-melanoma skin cancer (NMSC), e.g., superficial basal cell carcinoma (sBCC) and squamous cell carcinoma (SCC), and malignant melanoma.

In some embodiments, the use of compounds capable of activating NRF2 pathway related activity (e.g., through engagement with the Cys151 residue of the KEAP1 protein) within skin cells at risk for exposure to UV-radiation prevents pigment cells from pigment loss, prevents hair discoloration and/or hair aging, prevents vitiligo, and/or prevents skin damage related to solar tanning.

The invention also provides kits comprising any of the compositions of the present invention (e.g., composition comprising effective amounts of bixin) (e.g., compositions comprising effective amounts of compounds capable of activating NRF2 pathway related activity (e.g., through engagement with the Cys151 residue of the KEAP1 protein)) and instructions for administering the compositions to an animal. The kits may optionally contain other photoprotective agents, e.g., compositions comprising zinc oxide and/or titanium dioxide.

In some embodiments, the composition comprising an effective amount of bixin further comprises polyethylene glycol. In some embodiments, the amount of bixin within the composition comprising an effective amount of bixin and polyethylene glycol is approximately 1%. In some embodiments, activating NRF2 pathway related activity in the subject occurs in keratinocyte cells and/or pigment cells (e.g., melanocyte cells).

In certain embodiments, methods for preventing disorders related to skin barrier function are provided. Nrf2 signaling and the upregulation of Nrf2 target genes is now widely recognized as a major molecular factor underlying human skin barrier structure and function. Specifically, the established role of Nrf2 in keratinocyte biology indicates that Nrf2-directed molecular strategies that induce Nrf2 signaling will enhance skin barrier function by strengthening epidermal differentiation and thickness [58]. As such, in certain embodiments, methods for preventing disorders related to skin barrier function are provided involving cutaneous delivery of bixin and its derivatives (either systemically or topically) for purposes of enhancing skin barrier structure and function through Nrf2 modulation, providing therapeutic benefit in dermatologically relevant conditions that are associated with an impairment of skin barrier function including but not limited to atopic dermatitis, eczema, psoriasis, allergic skin inflammation, microbe-induced damage, and general chronological aging and senescence, all of which are characterized by diminished skin barrier function.

### BRIEF DESCRIPTION OF DRAWINGS

FIG. 1A-G: Bixin upregulates NRF2 signaling and antioxidant defenses in epidermal keratinocytes. (A) For Oxidative Stress RT² Profiler™ PCR Expression Array analysis, HEKs were exposed to bixin (20 μM, 24 h) followed by gene expression analysis; upper panel: scatter blot depiction of bixin-induced gene expression (versus untreated); cut-off lines: threefold up- or down-regulation; the insert shows the chemical structure of bixin; bottom panel: numerical expression changes [n=3, mean±SD; (p<0.05)]. (B) Bixin (0-20 μM, 0-24 h) increased the protein levels of NRF2 and its target genes as assessed by immunoblot analysis; left panel: dose-response, right panel: time course. (C) HaCaT keratinocytes cotransfected with NQO1-ARE firefly luciferase and Renilla luciferase reporters were treated with bixin (0-40 μM) for 16 h. Dual luciferase activities were measured; data are expressed as means±SD (*p<0.05, ctrl. vs. bixin treated groups). (D) HaCaT keratinocytes were treated with bixin (20 μM; 0-48 h exposure time), and cell lysates were subjected to immunoblot analysis. (E) HaCaT keratinocytes were treated with bixin (0-40 μM, 24 h), and total cellular glutathione was determined [n=3; means±SD (*p<0.05, ctrl. vs. bixin groups]. (F) HaCaT keratinocytes were exposed to bixin (20 μM; 1 and 24 h exposure time) followed by dye sensitization (generating ¹O₂) and subsequent loading with 2′,7′-dichlorodihydrofluorescein diacetate (DCFH-DA)]. Intracellular oxidative stress was then assessed by flow cytometric determination of DCF fluorescence intensity [means±SD, n=3; means without a common letter differ (p<0.05)]. (G) HaCaT keratinocytes were exposed to various anti-oxidants [1 h pretreatment: trolox (1 mM), tiron (500 μM), N-acetyl-L-cysteine (NAC; 10 mM)] followed by addition of bixin (40 μM; 4 h) and NRF2/KEAP1 immunoblot analysis.

FIG. 2A-C: Bixin causes Nrf2 activation without impairment of cell viability. (A) Bixin cytotoxicity in HaCaT cells (0-160 μM, 48 h continuous exposure; MTT assay; n=3; means±SD). Bixin-induced changes did not reach the level of statistical significance. (B) Bixin modulation of cell viability in response to bixin (40 μM; 48 h continuous exposure; annexinV/PI flow cytometry). The numbers indicate viable cells (AV⁻, PI⁻, lower left quadrant) in percent of total gated cells (n=3; means±SD). Bixin-induced changes did not reach the level of statistical significance. (C) HaCaT keratinocytes cells were treated with bixin (0-40 μM; 4 and 16 h) followed by immunoblot (NRF2/KEAP1/GAPDH) analysis.

FIG. 3A-G: Bixin induces KEAP1-C151-dependent NRF2 upregulation and increases Nrf2 protein half-life (t_{1/2}) in human keratinocytes. (A-D) HaCaT cells were either left untreated (control; empty bar) or treated with bixin (40 μM, filled bar; 4 h and 16 h), and mRNA was extracted. Relative mRNA levels [NRF2 (A), KEAP1 (B), GCLM(C), AKR1C1 (D)] as determined by quantitative real-time RT-PCR [means±SD (*p<0.05, control vs. bixin treated group)]. (E) HaCaT cells were either left untreated or treated with bixin (40 μM, 4 h). Cycloheximide (CHX, 50 μM) was added and cells were lysed at the indicated time points followed by immunoblot analysis using NRF2 and GAPDH antibodies. Band intensities were quantified and plotted against the time after CHX treatment to obtain half-life (t_{1/2}) values. (F) HaCaT cells were cotransfected with plasmids encoding the indicated proteins; 24 h later the cells were then left untreated or treated with either SF (5 μM) or bixin (40 μM) along with MG132 (10 μM) for 4 h. Anti-NRF2 immunoprecipitates were analyzed by immunoblotting with anti-HA antibody detecting ubiquitin-conjugated NRF2. (G) HaCaT cells cotransfected with the plasmids expressing either wild type KEAP1 (KEAP1-WT) or C151 mutated KEAP1 (KEAP1-C151S) along with NQO1-ARE firefly luciferase and Renilla luciferase reporters were left untreated or treated with the indicated compounds (16 h). Dual luciferase activities were measured; data are expressed as means±SD (*p<0.05, Control vs. compound treated groups; ^{#}p<0.05, KEAP1-WT vs. KEAP1-C151S group.)

FIG. 4A-D: Plasma kinetics and cutaneous NRF2 modulation after systemic administration of bixin in SKH-1 mice. Bixin analysis in mouse plasma: (A) HPLC chromatogram (10 μg/ml plasma) with photodiode array detection (B; 300-580 nm absorbance; peak absorbance as indicated numerically). (C) Mouse plasma (Nrf2^{+/+} versus Nrf2^{-/-}) was collected at various time points (0, 1, 2, 4, 8, 16, 24, 48, 72 h) after compound (200 mg/kg; i.p.) administration, and bixin plasma levels (μg/ml) were determined (n=3; means±SD). (D) At various time points (48, 72 h) after bixin systemic administration (up to 200 mg/kg), skin tissue was harvested and lysates from Nrf2^{+/+} mice were subjected to immunoblot analyses (NRF2, KEAP1, AKR1C1, GCLM, and GAPDH; n=2, each lane represents an individual mouse).

FIG. 5A-D: Systemic administration of bixin activates cutaneous NRF2 and NRF2 targets. Mice (Nrf2^{+/+} and Nrf2^{-/-} mice; n=6 per group) received bixin treatment (200 mg/kg; i.p.) or carrier control (corn oil), followed by solar UV (UVB 240 mJ/cm²) or mock exposure performed 48 h after bixin administration. (A) After UV exposure (24 h), IHC analysis (NRF2, GCLM, AKR1C1) was performed using skin tissue sections; representative tissue from each group is shown (scale bar: 100 μm). (B) Skin tissue lysates from Nrf2^{+/+} mice were subjected to immunoblot analyses with anti-NRF2, KEAP1, AKR1C1, GCLM, and GAPDH antibodies (n=3, each lane represents an individual mouse). (C-D) Skin prepared from mice as specified in (A) was processed for determination of mRNA levels [Gclm (C) and Akr1c1 (D)] using quantitative RT-PCR; means±SD (*p<0.05, control vs. treatment groups).

FIG. 6A-B: Systemic administration of bixin causes NRF2 activation in SKH-1 murine skin without changing Nrf2 or Keap1 mRNA levels. As described in FIG. 3, mice (Nrf2^{+/+} and Nrf2^{-/-} mice; n=6 per group) received bixin treatment (200 mg/kg; i.p.) or carrier control (corn oil), followed by solar UV (UVB 240 mJ/cm²) or mock exposure applied 48 h after bixin. After irradiation (24 h), skin was processed for determination of mRNA levels [Nrf2 (upper panel) and Keap1 (upper)] using quantitative RT-PCR; means±SD (*p<0.05).

FIG. 7A-C: Systemic administration of bixin suppresses UV-induced epidermal thickening, apoptosis, and oxidative DNA damage in Nrf2^{+/+} mice but not Nrf2^{-/-} mice. Mice (Nrf2^{+/+} and Nrf2^{-/-} mice; n=6 per group) received bixin treatment (200 mg/kg; i.p.) or carrier control (corn oil), followed by solar UV (UVB 240 mJ/cm²) or mock exposure performed 48 h after bixin. (A) After irradiation (24 h), H&E staining and in situ TUNEL analysis visualizing epidermal apoptotic cells were performed [n=6; representative tissue from each group is shown (scale bar: 100 μm)]. In addition, 8-oxo-dG- and CPD-lesions were visualized by IHC; representative tissue from each group is shown. (B) Epidermal thickness in H&E-stained sections was measured as the distance between the top of the basement membrane and the bottom of the stratum corneum at five randomly selected fields from each mouse specimen. (C) Quantification of TUNEL-positive cells (green fluorescent nuclei) in five random fields per section; 200× magnification; [means±SD (*p<0.05, control vs. treatment groups; ^{#}p<0.05, UV vs. bixin+UV groups)].

FIG. 8A-E: Systemic administration of bixin attenuates UV-induced cutaneous hyperproliferation and inflammation in Nrf2^{+/+} mice but not Nrf2^{-/-} mice. Mice were treated as detailed in FIGS. 3 and 4 followed by IHC analysis for (A) Ki67 and (B) MMP9 (scale bar: 100 pin). (C-D) Skin tissue lysates from bixin/UV-exposed Nrf2^{+/+} and Nrf2^{-/-} mice were also subjected to immunoblot analyses with anti-p-p65, p65, and GADPH antibodies followed by quantification using densitometry (D). (E) mRNA levels of IL6, TNFα and MMP9 were determined using quantitative RT-PCR. Results are expressed as means±SD (*p<0.05, control vs. treatment groups; ^{#}p<0.05, UV vs. bixin+UV groups).

FIG. 9A shows 1% Bixin in a standard topical carrier (Vanicream) is not efficient in upregulating cutaneous Nrf2. SKH-1 mice were treated with topical Bixin (1% in Vanicream carrier; 50 μl per application) (‘Bixin’).

FIG. 9B shows that 1% Bixin in PEG400 (e.g., polyethylene glycol; average mass 400 Da) is very efficient in upregulating the Nrf2-dependent cytoprotective response with topical administration.

FIG. 10A shows that Bixin is an efficient Nrf2 activator in human skin melanocytes.

FIG. 10B shows that Bixin is an efficient activator of cytoprotective Nrf2 target gene expression in human skin melanocytes.

FIG. 11 shows that UV-exposure of Bixin enhances bixin potency as an activator of Nrf2 target gene expression in human skin keratinocytes.

### DETAILED DESCRIPTION OF THE INVENTION

Exposure to solar ultraviolet (UV) radiation is a causative factor in skin photodamage and carcinogenesis [1-3]. Even though sunscreen-based broad-spectrum photoprotection is an effective key component of a sun-safe strategy to reduce cumulative lifetime exposure to UV light, much effort has been directed towards the development of more effective molecular strategies for cutaneous photoprotectants acting through mechanisms different from (or synergistic with) photon absorption [4-7].

The redox-sensitive transcription factor NRF2 (nuclear factor-E2-related factor 2) orchestrates major cellular defense mechanisms including phase-II detoxification, inflammatory signaling, DNA repair, and antioxidant response, and NRF2 has therefore emerged as a promising molecular target for the pharmacological prevention of human pathologies resulting from exposure to environmental toxicants including solar UV light [8-11]. Recent studies strongly suggest a protective role of NRF2-mediated gene expression in the suppression of cutaneous photodamage induced by solar UV radiation, and NRF2 activation has been shown to protect cutaneous keratinocytes and fibroblasts against the cytotoxic effects of UVA and UVB [9, 11-21]. Importantly, recent research performed in SKH-1 mice documents that constitutive genetic NRF2 activation protects mice against acute photodamage and photocarcinogenesis [22]. Therefore, pharmacological modulation of NRF2 has now attracted considerable attention as a novel approach to skin photoprotection [19, 21, 23]. Indeed, protection of primary human keratinocytes from UVB-induced cell death by novel drug-like NRF2 activators has been reported, a photoprotective effect attributed in part to NRF2-dependent elevation of cellular glutathione levels [20, 24, 25]. Topical application of NRF2 inducers, e.g. the synthetic NRF2-activator TBE-31, has shown pronounced photoprotective and photochemopreventive activity in murine skin, and suppression of solar UV-induced human skin erythema was achieved by topical application of a standardized broccoli extract delivering the NRF2 inducer sulforaphane [22]; however, there has been little research exploring the concept of cutaneous photoprotection and photochemoprevention achieved by systemic administration of NRF2 inducers [26].

Dietary carotenoids (including β-carotene, lycopene, lutein, 3,3′-dihydroxyisorenieratene, zeaxanthin, astaxanthin) and their biosynthetic precursor molecules (such as phytoene) have been under investigation for cutaneous photoprotection, and feasibility of carotenoid-based nutritional photoprotection has been demonstrated in murine and human skin [4, 27-30]. The systemic photoprotective activity of carotenoids, displayed only after dietary uptake and cutaneous accumulation, has largely been attributed to their activity as photon absorbers, sacrificial antioxidants, and excited state/singlet oxygen quenchers [30-32]. However, the mechanistic involvement of NRF2 activation in carotenoid-based systemic photoprotection has not been investigated before.

In an attempt to test the feasibility of NRF2-dependent systemic photoprotection by dietary constituents, experiments conducted during the course of developing embodiments for the present invention conducted photoprotection studies on the apocarotenoid bixin, an FDA-approved natural food colorant from the seeds of the achiote tree (Bixa orellana) native to tropical America [33, 34]. Consumed by humans since pre-Columbian times, this apocarotenoid derived from lycopene through oxidative cleavage is now used worldwide as a dietary additive and cosmetic ingredient (referred to as ‘annato’; E160b) with an excellent safety record and established systemic bioavailability and pharmacokinetic profile upon oral administration [35-37]. Such experiments demonstrated that (i) bixin is a potent activator of the NRF2-dependent cytoprotective response in cultured human skin keratinocytes, that (ii) systemic administration of bixin activates cutaneous NRF2 with potent protective effects against solar UV-induced skin damage in SKH-1 mice, that (iii) bixin-induced suppression of photodamage is observable in Nrf2^{+/+} but not in Nrf2^{-/-} SKH-1 mice confirming the NRF2-dependence of bixin-based antioxidant and anti-inflammatory cutaneous effects, (iv) bixin activates NRF2 through the critical Cys-151 sensor residue in KEAP1, orchestrating a broad cytoprotective response in cultured human keratinocytes as revealed by antioxidant gene expression array analysis, (v) that administration of 1% bixin in PEG based carrier activates Nrf2 and Nrf2 target expression in skin tissues of SKH-1 mice, but not in a standard topical carrier (e.g., Vanicream), (vi) that bixin treatment induces Nrf2 and Nrf2 target gene expression in human primary skin melanocytes, and (vii) that irradiation of bixin with solar ultraviolet light enhances (‘potentiates’) bixin activity for upregulation of cytoprotective gene expression in human skin keratinocytes.

Accordingly, provided herein are methods for preventing conditions related to UV-radiation exposure in subjects at risk for exposure to UV-radiation. In particular, the invention relates to compositions comprising specific formulations of dietary carotenoids (e.g., bixin) which function as activators of NRF2 pathway related activity, and related methods for the protection of mammalian skin against UV-radiation.

As noted, the Applicants have found that effective amounts of the apocarotenoid bixin

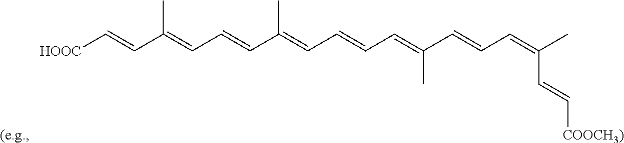

functions as an activator of NRF2 pathway related activity (e.g., through engagement with the Cys151 residue of the KEAP1 protein) within skin cells at risk for exposure to UV-radiation.

The invention also provides pharmaceutical compositions comprising the compounds of the invention in a pharmaceutically acceptable carrier. As such, in certain embodiments, the present invention provides pharmaceutical compositions comprising effective amounts of the apocarotenoid bixin, or variants thereof, wherein the composition has cutaneous photoprotective properties against A or B forms of ultraviolet radiation.

As noted, experiments conducted during the course of developing embodiments for the present invention demonstrated that irradiation of bixin with solar ultraviolet light enhances (potentiates′) bixin activity for upregulation of cytoprotective gene expression in human skin keratinocytes. As such, in some embodiments, the bixin is irradiated bixin. In some embodiments, the source of irradiation is selected from ultraviolet light, visible light, and ionizing radiation. In some embodiments, the result of irradiation is the formation of photochemical bixin derivatives and degradation products.

In some embodiments, the pharmaceutical composition functions as an activator of NRF2 pathway related activity (e.g., through engagement with the Cys151 residue of the KEAP1 protein) within skin cells at risk for exposure to UV-radiation. In some embodiments, effective amounts of compounds structurally similar to bixin (e.g., norbixin) which also are capable of activating NRF2 pathway related activity (e.g., through engagement with the Cys151 residue of the KEAP1 protein) within skin cells at risk for exposure to UV-radiation are provided. In some embodiments, any compound capable of activating NRF2 pathway related activity (e.g., through engagement with the Cys151 residue of the KEAP1 protein) within skin cells at risk for exposure to UV-radiation is provided. In some embodiments, the administration resulting in activation of NRF2 pathway related activity in skin cells including, but not limited to, keratinocyte cells and/or pigment cells (e.g., melanocyte cells).

Such compounds may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

The invention further provides processes for preparing any of the compounds of the present invention.

The invention also provides the use of compounds capable of activating NRF2 pathway related activity (e.g., through engagement with the Cys151 residue of the KEAP1 protein) within skin cells at risk for exposure to UV-radiation (e.g., bixin and compound structurally similar to bixin) for purposes of protecting skin from conditions caused by UV-radiation exposure. Examples of conditions caused by UV-radiation exposure include, but are not limited to, advanced skin aging or wrinkling, thickening of the skin (e.g., the leathery, weather-beaten, elephant hide look), uneven or pebbly skin, flabbiness, lifeless skin, pigmentation irregularities, small dilated blood vessels or red markings on or near the surface of the skin also known as telangiectasias, rough or scaly patches, e.g., actinic keratoses, freckles otherwise known as ephilides, liver spots, age spots, dark spots or skin tags known as lentigines, pre-skin cancers, and skin cancer, such as non-melanoma skin cancer (NMSC), e.g., superficial basal cell carcinoma (sBCC) and squamous cell carcinoma (SCC), and malignant melanoma.

Such methods require that the amount of bixin delivered to the subject be sufficient to activate the NRF2 pathway related activity (e.g., through engagement with the Cys151 residue of the KEAP1 protein) within skin cells at risk for exposure to UV-radiation. In some embodiments, the dosage amount of bixin is approximately between 10 mg/kg and 200 mg/kg of the subject. Indeed, experiments conducted during the course of developing embodiments for the present invention determined that dosage amounts less than 10 mg/kg of the subject were insufficient to activate the NRF2 pathway related activity (e.g., through engagement with the Cys151 residue of the KEAP1 protein) within skin cells at risk for exposure to UV-radiation.

Such methods are not limited to a particular manner of administering the composition comprising bixin.

In some embodiments, the composition comprising bixin is administered topically in a skin area at risk for exposure to UV-radiation (e.g., in the form of a cream, gel, oil, or lotion). In some embodiments involving topical administration, the bixin is within a composition further comprising polyethylene glycol. In such embodiments, the amount of bixin within a composition comprising bixin and polyethylene glycol is approximately 1% (e.g., 0.5%, 0.7%, 0.85%, 0.9%, 0.95%, 0.999%, 1%, 1.05%, 1.1%, 1.5%, 1.75%, 2%, 2.5%, etc.).

In some embodiments, the composition comprising bixin is orally administered to achieve systemic administration. Indeed, the manner of administration is irrelevant so long as the resulting administration results in activation of NRF2 pathway related activity (e.g., through engagement with the Cys151 residue of the KEAP1 protein) within skin cells at risk for exposure to UV-radiation.

In certain embodiments of the invention, combination prophylactic treatment of animals at risk for UV-radiation skin damage with a therapeutically effective amount of a composition comprising bixin and a course of an additional photoprotective agent known to prevent UV-radiation related skin damage produces a greater prevention of UV-radiation related skin damage and clinical benefit in such animals compared to those treated with agent known to prevent UV-radiation related skin damage (e.g., the additional photoprotective agent alone. In some embodiments, the composition comprising bixin is a part of a larger composition known to prevent UV-radiation related skin damage (e.g., a part of the additional photoprotective agent). Examples of additional protective agents include, but are not limited to, sun screen, sunblock, suntan lotion, sunburn cream, sun cream and block out. In some embodiments, the additional photoprotective agent is a composition comprising effective amounts of titanium dioxide. In some embodiments, the additional photoprotective agent is a composition comprising effective amounts of zinc oxide. In some embodiments, the additional photoprotective agent is a composition comprising effective amounts of titanium dioxide and zinc oxide. In some embodiments, the additional photoprotective agent is a composition comprising effective amounts of one or more of the following: p-aminobenzoic acid, padimate 0, phenylbenzimidazole sulfonic acid, cinoxate, dioxybenzone, oxybenzone, homosalate, menthyl anthranilate, octocrylene, octyl methoxycinnamate, octyl salicylate, sulisobenzone, trolamine salicylate, avobenzone, ecamsule, titanium dioxide, zinc oxide, 4-methylbenzylidene camphor, tinosorb M, tinosorb S, tinosorb A2B, neo heliopan AP, mexoryl XL, benzophenone-9, uvinul T 150, uvinul A Plus, uvasorb HEB, parsol SLX, and amiloxate.

The invention also provides kits comprising any of the compositions of the present invention (e.g., composition comprising effective amounts of bixin) (e.g., compositions comprising effective amounts of compounds capable of activating NRF2 pathway related activity (e.g., through engagement with the Cys151 residue of the KEAP1 protein)) and instructions for administering the compositions to an animal. The kits may optionally contain other photoprotective agents, e.g., compositions comprising zinc oxide and/or titanium dioxide.

In some embodiments, the composition comprising an effective amount of bixin further comprises polyethylene glycol. In some embodiments, the amount of bixin within the composition comprising an effective amount of bixin and polyethylene glycol is approximately 1%. In some embodiments, activating NRF2 pathway related activity in the subject occurs in keratinocyte cells and/or pigment cells (e.g., melanocyte cells).

In some embodiments, the methods involving the administration of compositions of the present invention (e.g., composition comprising effective amounts of bixin) (e.g., compositions comprising effective amounts of compounds capable of activating NRF2 pathway related activity (e.g., through engagement with the Cys151 residue of the KEAP1 protein)) further involve co-administration with an anticancer agent.

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics;

antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a compound of the invention and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present invention include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 2 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 2

| | | |
|---|---|---|
| Aldesleukin | Proleukin | Chiron Corp., Emeryville, |
| (des-alanyl-1, serine-125 human interleukin-2) | | CA |
| Alemtuzumab | Campath | Millennium and ILEX |
| (IgG1κ anti CD52 antibody) | | Partners, LP, Cambridge, |
| | | MA |
| Alitretinoin | Panretin | Ligand Pharmaceuticals, |
| (9-cis-retinoic acid) | | Inc., San Diego CA |
| Allopurinol | Zyloprim | GlaxoSmithKline, |
| (1,5-dihydro-4 H-pyrazolo[3,4-d]pyrimidin-4- | | Research Triangle Park, |
| one monosodium salt) | | NC |
| Altretamine | Hexalen | US Bioscience, West |
| (N,N,N′,N′,N″,N″,-hexamethyl-1,3,5-triazine-2, | | Conshohocken, PA |
| 4, 6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a, a, a′, a′- | | Pharmaceuticals, LP, |
| tetramethyl-5-(1H-1,2,4-triazol-1-ylmethyl)) | | Wilmington, DE |
| Arsenic trioxide | Trisenox | Cell Therapeutic, Inc., |
| | | Seattle, WA |
| Asparaginase | Elspar | Merck & Co., Inc., |
| (L-asparagine amidohydrolase, type EC-2) | | Whitehouse Station, NJ |
| BCG Live | TICE | Organon Teknika, Corp., |
| (lyophilized preparation of an attenuated strain | BCG | Durham, NC |
| of Mycobacterium bovis (Bacillus Calmette- | | |
| Gukin [BCG], substrain Montreal) | | |
| bexarotene capsules | Targretin | Ligand Pharmaceuticals |
| (4-[1-(5,6,7,8-tetrahydro-3,5,5,8,8-pentamethyl- | | |
| 2-napthalenyl) ethenyl] benzoic acid) | | |
| bexarotene gel | Targretin | Ligand Pharmaceuticals |
| Bleomycin | Blenoxane | Bristol-Myers Squibb Co., |
| (cytotoxic glycopeptide antibiotics produced by | | NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0, 0′]-,(SP-4-2)) | | |
| Carmustine | BCNU, | Bristol-Myers Squibb |
| (1,3-bis(2-chloroethyl)-1-nitrosourea) | BiCNU | |
| Carmustine with Polifeprosan 20 Implant | Gliadel | Guilford Pharmaceuticals, |
| | Wafer | Inc., Baltimore, MD |
| Celecoxib | Celebrex | Searle Pharmaceuticals, |
| (as 4-[5-(4-methylphenyl)-3-(trifluoromethyl)- | | England |
| 1H-pyrazol-1-yl] | | |
| benzenesulfonamide) | | |
| Chlorambucil | Leukeran | GlaxoSmithKline |
| (4-[bis(2chlorethyl)amino]benzenebutanoic | | |
| acid) | | |
| Cisplatin | Platinol | Bristol-Myers Squibb |
| (PtCl₂H₆N₂) | | |
| Cladribine | Leustatin, | R.W. Johnson |
| (2-chloro-2′-deoxy-b-D-adenosine) | 2-CdA | Pharmaceutical Research |
| | | Institute, Raritan, NJ |
| Cyclophosphamide | Cytoxan, | Bristol-Myers Squibb |
| (2-[bis(2-chloroethyl)amino] tetrahydro-2H- | Neosar | |
| 13,2-oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, Inc., |
| | | San Diego, CA |
| Dacarbazine | DTIC- | Bayer AG, Leverkusen, |
| (5-(3,3-dimethyl-1-triazeno)-imidazole-4- | Dome | Germany |
| carboxamide (DTIC)) | | |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., Thousand |
| (recombinant peptide) | | Oaks, CA |
| daunorubicin liposomal | DanuoXome | Nexstar Pharmaceuticals, |
| ((8S-cis)-8-acetyl-10-[(3-amino-2,3,6-trideoxy- | | Inc., Boulder, CO |
| á-L-lyxo-hexopyranosyl)oxyl-7,8,9,10- | | |
| tetrahydro-6,8,11-trihydroxy-1-methoxy-5,12- | | |
| naphthacenedione hydrochloride) | | |
| Daunorubicin HCl, daunomycin | Cerubidine | Wyeth Ayerst, Madison, |
| ((1 S ,3 S )-3-Acetyl-1,2,3,4,6,11-hexahydro- | | NJ |
| 3,5,12-trihydroxy-10-methoxy-6,11-dioxo-1- | | |
| naphthacenyl 3-amino-2,3,6-trideoxy-(alpha)-L- | | |
| lyxo-hexopyranoside hydrochloride) | | |
| Denileukin diftitox | Ontak | Seragen, Inc., Hopkinton, |
| (recombinant peptide) | | MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis Pharmaceuticals, |
| ((2R,3S)-N-carboxy-3-phenylisoserine, N-tert- | | Inc., Bridgewater, NJ |
| butyl ester, 13-ester with 5b-20-epoxy- | | |
| 12a,4,7b,10b,13a-hexahydroxytax-11-en-9-one | | |
| 4-acetate 2-benzoate, trihydrate) | | |
| Doxorubicin HCl | Adriamycin, | Pharmacia & Upjohn |
| (8S,10S)-10-[(3-amino-2,3,6-trideoxy-a-L-lyxo- | Rubex | Company |
| hexopyranosyl)oxy]-8-glycolyl-7,8,9,10- | | |
| tetrahydro-6,8,11- trihydroxy-1-methoxy-5,12- | | |
| naphthacenedione hydrochloride) | | |
| doxorubicin | Adriamycin | Pharmacia & Upjohn |
| | PFS | Company |
| | Intravenous | |
| | injection | |
| doxorubicin liposomal | Doxil | Sequus Pharmaceuticals, |
| | | Inc., Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo Alto, |
| | injection | CA |
| Elliott's B Solution | Elliott's B | Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino- hexopyranosyl)oxyl-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3- | | Company |
| [bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3, 17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, Inc., |
| (fluorinated nucleotide analog of the antiviral | | Cedar Knolls, NJ |
| agent vidarabine, 9-b-D- | | |
| arabinofuranosyladenine (ara-A)) | | |
| Fluorouracil, 5-FU | Adrucil | ICN Pharmaceuticals, Inc., |
| (5-fluoro-2,4(1H,3H)-pyrimidinedione) | | Humacao, Puerto Rico |
| Fulvestrant | Faslodex | IPR Pharmaceuticals, |
| (7-alpha-[9-(4,4,5,5,5-penta | | Guayama, Puerto Rico |
| fluoropentylsulphinyl) nonyl]estra-1,3,5-(10)- | | |
| triene-3,17-beta-diol) | | |
| Gemcitabine | Gemzar | Eli Lilly |
| (2′-deoxy-2′, 2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex | AstraZeneca |
| | Implant | Pharmaceuticals |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal antibody | | |
| Ibritumomab and the linker-chelator titmetan | | |
| [N-[2-bis(carboxymethyl)amino]-3-(p- | | |
| isothiocyanatophenyl)-propyl]-[N-[2- | | |
| bis(carboxymethyl)amino]-2-(methyl) - | | |
| ethyl]glycine) | | |
| Idarubicin | Idamycin | Pharmacia & Upjohn |
| (5, 12-Naphthacenedione, 9-acetyl-7-[(3-amino- | | Company |
| 2,3,6-trideoxy-(alpha)-L-lyxo- | | |
| hexopyranosyl)oxy]-7,8,9,10-tetrahydro-6,9,11- | | |
| trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La Roche, Inc., |
| (recombinant peptide) | | Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperi- | | Company |
| dinopiperidino)carbonyloxy]-1H-pyrano[3′, 4′: | | |
| 6,7] indolizino[1,2-b] quinoline-3,14(4H, 12H) | | |
| dione hydrochloride trihydrate) | | |
| Letrozole | Femara | Novartis |
| (4,4′-(1H-1,2,4 -Triazol-1-ylmethylene) | | |
| dibenzonitrile) | | |
| Leucovorin | Wellcovorin, | Immunex, Corp., Seattle, |
| (L-Glutamic acid, N[4[[(2amino-5-formyl- | Leucovorin | WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((-)-( S)-2,3,5, 6-tetrahydro-6-phenylimidazo | | Foundation, Titusville, NJ |
| [2,1-b] thiazole monohydrochloride | | |
| C₁₁H₁₂N₂S•HCl) | | |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers Squibb |
| 17α( acetyloxy)-6-methylpregna-4,6-diene- | | |
| 3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6 H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7- | | Exton, Pa |
| one) | | |
| Mitomycin C | Mutamycin | Bristol-Myers Squibb |
| mitomycin C | Mitozytrex | SuperGen, Inc., Dublin, |
| | | CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, Germany |
| Oprelvekin | Neumega | Genetics Institute, Inc., |
| (IL-11) | | Alexandria, VA |
| Oxaliplatin | Eloxatin | Sanofi Synthelabo, Inc., |
| (cis-[(1R,2R)-1,2-cyclohexanediamine-N,N′] | | NY, NY |
| [oxalato(2-)-O,O′] platinum) | | |
| Paclitaxel | TAXOL | Bristol-Myers Squibb |
| (5ß, 20-Epoxy-1,2a, 4,7ß, 10ß, 13a- | | |
| hexahydroxytax-11-en-9-one 4,10-diacetate 2- | | |
| benzoate 13-ester with (2R, 3 S)-N-benzoyl-3- | | |
| phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen | Enzon Pharmaceuticals, |
| ((monomethoxypolyethylene glycol | (Pegademase | Inc., Bridgewater, NJ |
| succinimidyl) 11-17-adenosine deaminase) | Bovine) | |
| Pegaspargase | Oncaspar | Enzon |
| (monomethoxypolyethylene glycol succinimidyl | | |
| L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, |
| | | Inc., Vancouver, |
| | | Canada |
| Procarbazine | Matulane | Sigma Tau |
| (N-isopropyl-μ-(2-methylhydrazino)-p- | | Pharmaceuticals, Inc., |
| toluamide monohydrochloride) | | Gaithersburg, MD |
| Quinacrine | Atabrine | Abbott Labs |
| (6-chloro-9-( 1-methyl-4-diethyl-amine) | | |
| butylamino-2-methoxyacridine) | | |
| Rasburicase | Elitek | Sanofi-Synthelabo, Inc., |
| (recombinant peptide) | | |
| Ritimimab | Rituxan | Genentech, Inc., South San |
| (recombinant anti-CD20 antibody) | | Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and | | |
| b)-D-glucopyranose and 220 mg citric acid | | |
| anhydrous) | | |
| Talc | Sclerosol | Bryan, Corp., Woburn, MA |
| (Mg₃Si₄O₁₀ (OH)₂) | | |
| Tamoxifen | Nolvadex | AstraZeneca |
| ((Z)2-[4-(1,2-diphenyl-1-butenyl) phenoxy]-N, | | Pharmaceuticals |
| N-dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4- | | |
| dien-17-oic acid [dgr ]-lactone) | | |
| Thioguanine, 6-TG | Thioguanine | GlaxoSmithKline |
| (2-amino-1,7-dihydro-6 H-purine-6-thione) | | |
| Thiotepa | Thioplex | Immunex Corporation |
| (Aziridine, 1,1′,1″-phosphinothioylidynetris-, or | | |
| Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′, 4′: 6,7] indolizino | | |
| [1,2-b] quinoline-3,14-(4H,12H)-dione | | |
| monohydrochloride) | | |
| Toremifene | Fareston | Roberts Pharmaceutical |
| (2-(p-[(Z)-4-chloro-1,2-diphenyl-1-butenyl]- | | Corp., Eatontown, NJ |
| phenoxy)-N,N-dimethylethylamine citrate (1:1)) | | |
| Tositumomab, I 131 Tositumomab | Bexxar | Corixa Corp., Seattle, WA |
| (recombinant murine immunotherapeutic | | |
| monoclonal IgG₂ₐ lambda anti-CD20 antibody (I | | |
| 131 is a radioimmunotherapeutic antibody)) | | |
| Trastuzumab | Herceptin | Genentech, Inc |
| (recombinant monoclonal IgG₁ kappa anti- | | |
| HER2 antibody) | | |
| Tretinoin, ATRA | Vesanoid | Roche |
| (all-trans retinoic acid) | | |
| Uracil Mustard | Uracil | Roberts Labs |
| | Mustard | |
| | Capsules | |
| Valrubicin, N-trifluoroacetyladriamycin-14- | Valstar | Anthra --> Medeva |
| valerate | | |
| ((2S-cis)-2- [1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2- | | |
| oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, 06-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

The present invention provides methods for administering the compositions of the present invention (e.g., composition comprising effective amounts of bixin) (e.g., compositions comprising effective amounts of compounds capable of activating NRF2 pathway related activity (e.g., through engagement with the Cys151 residue of the KEAP1 protein)) with radiation therapy for purposes of preventing radiation related skin burning. The invention is not limited by the types, amounts, or delivery and administration systems used to deliver the therapeutic dose of radiation to an animal. For example, the animal may receive photon radiotherapy, particle beam radiation therapy, other types of radiotherapies, and combinations thereof. In some embodiments, the radiation is delivered to the animal using a linear accelerator. In still other embodiments, the radiation is delivered using a gamma knife.

The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by animals. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

Any type of radiation can be administered to an animal, so long as the dose of radiation is tolerated by the animal without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581). The effects of radiation can be at least partially controlled by the clinician. In one embodiment, the dose of radiation is fractionated for maximal target cell exposure and reduced toxicity.

Compositions within the scope of this invention include all compositions wherein the amount of bixin (or related variants) are contained in an amount which is effective to achieve its intended purpose (e.g., effective amounts of compounds capable of activating NRF2 pathway related activity (e.g., through engagement with the Cys151 residue of the KEAP1 protein)). While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being at risk for UV-radiation exposure. In one embodiment, compositions comprising about 0.01 to about 25 mg/kg bixin is orally administered to prevent UV-radiation related skin damage. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the bixin (or variants thereof) into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compositions of the invention. Foremost among such patients are mammals, e.g., humans, although the invention is not intended to be so limited. Other patients include veterinary animals at risk for UV-radiation related skin damage.

The pharmaceutical compositions may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example 1

This example demonstrates that bixin activates NRF2 and NRF2 target gene expression with upregulation of antioxidant defenses in human keratinocytes.

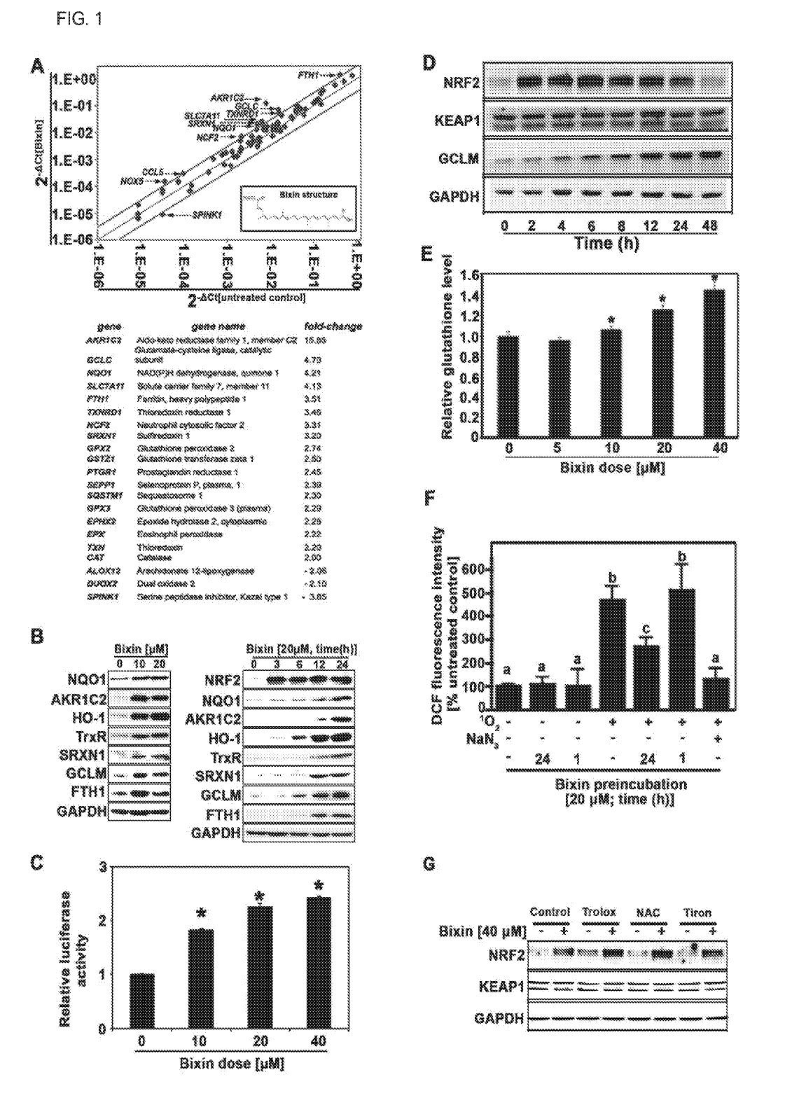

First, to comprehensively monitor antioxidant response gene expression induced by bixin (20 μM, 24 h) in primary human epidermal keratinocytes (HEKs) Oxidative Stress RT² Profiler™ PCR Expression Array analysis was performed (FIG. 1A). Pronounced upregulation of established NRF2 target genes involved in antioxidant protection and redox homeostasis (including AKR1C2, GCLC, NQO1, SLC7A11, FTH1, TXNRD1, NCF2, SRXN). Immunoblot analysis confirmed NRF2 activation in HEKs in response to bixin treatment as evident from increased protein levels of NRF2 and NRF2 targets including NQO1, AKR1C2, HO-1, TrxR, GCLM, SRXN1, and FTH1 (FIG. 1B left: ≤20 μM, 24 h; FIG. 1B right: exposure time ≤24 h, bixin 20 μM).

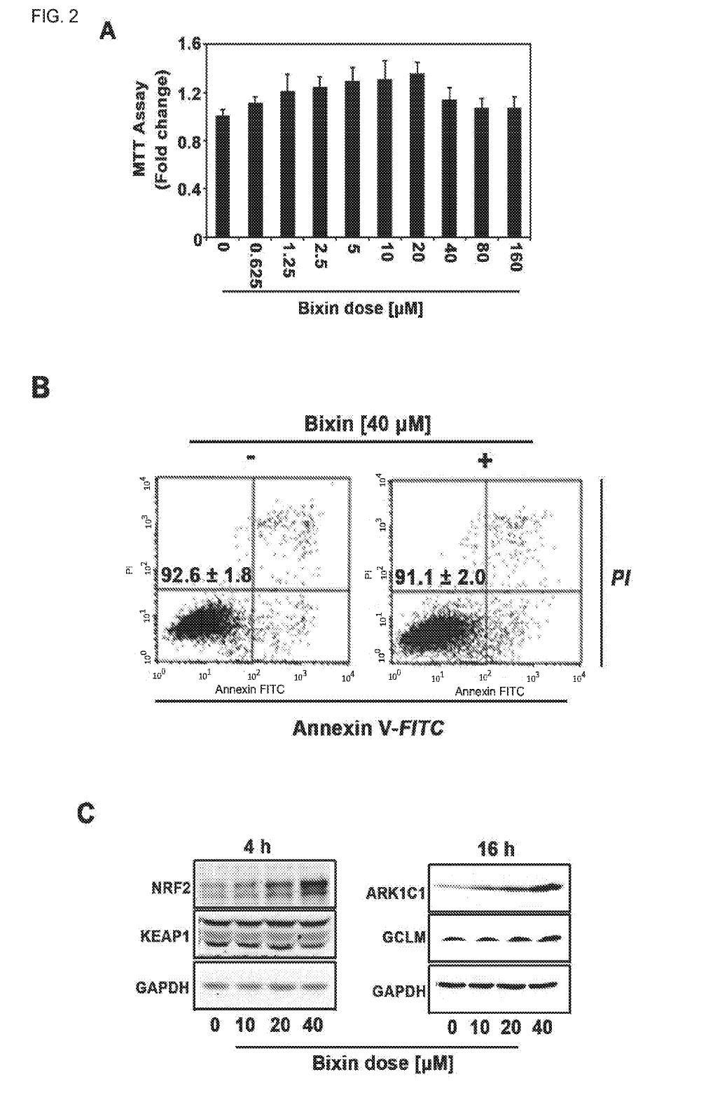

Next, the molecular mechanism underlying NRF2 activation by bixin was investigated in immortalized human HaCaT keratinocytes. Employing a dual luciferase ARE-reporter assay, dose-dependent induction of NRF2 transcriptional activity was elicited by bixin treatment, observable at concentrations (10-40 μM) devoid of cytotoxicity as detected employing the photometric MTT assay (FIG. 2A) and flow cytometric assessment of annexinV-PI stained cells (FIG. 2B). Time course analysis revealed a rapid induction of NRF2 protein levels detectable within 2 h treatment, while no changes in KEAP1 protein levels were observed (FIG. 1D). Moreover, NRF2 target proteins including GCLM and AKR1C1 (FIG. 1D and FIG. 2C) were upregulated in response to bixin treatment, and bixin-based upregulation of NRF2 was sustained over the course of the 24 h treatment, whereas upregulation of GCLM persisted over an extended period (48 h; FIG. 1D). Consistent with upregulation of glutathione biosynthesis factors (GCLM), total cellular glutathione was increased by almost 50% in response to bixin exposure (FIG. 1E). Since it has already been established that bixin displays potent activity as a direct physical singlet oxygen (¹O₂) quencher [30-32], if bixin pretreatment was able to protect HaCaT cells against ¹O₂-induced photo-oxidative stress was examined (FIG. 1F). Using flow cytometric detection of DCF fluorescence intensity [after ¹O₂-exposed cells were loaded with 2′,7′-dichlorodihydrofluorescein diacetate (DCFH-DA)], it was observed that bixin (20 μM) pretreatment efficiently suppressed the almost five fold increase in DCF fluorescence elicited by ¹O₂ originating from dye sensitization (employing an established toluidine blue/visible light-based regimen) [14, 44]. Remarkably, only prolonged preincubation (24 h) with bixin (20 μM) protected HaCaT cells against oxidative stress originating from dye sensitization. In contrast, shorter preincubation periods (1 h) were without protective effects, an observation consistent with the mechanistic involvement of bixin-induced upregulation of cellular antioxidant defenses underlying cytoprotective effects against photooxidative stress. However, the extent of bixin-induced cytoprotection did not reach the level of cytoprotective efficacy displayed by the physical ¹O₂-quencher NaN₃ (10 mM), active only if present during visible light-driven dye sensitization (FIG. 1F). It was also observed that exposure to bixin only (1 or 24 h exposure time) did not cause an increase in DCF fluorescence (FIG. 1F). Moreover, bixin-induced upregulation of Nrf2 protein levels was not attenuated by cotreatment with various anti-oxidants [tiron, trolox, N-acetyl-L-cysteine (NAC); FIG. 1G][46]. Likewise, 24 h preincubation using NAC did not attenuate bixin-induced Nrf2 upregulation, indicating that bixin does not cause Nrf2 upregulation through induction of intracellular oxidative stress.

### Example 2

This example demonstrates that bixin activates NRF2 in a KEAP1-C151 dependent manner and increases Nrf2 protein half-life (t_{1/2}) in human keratinocytes.

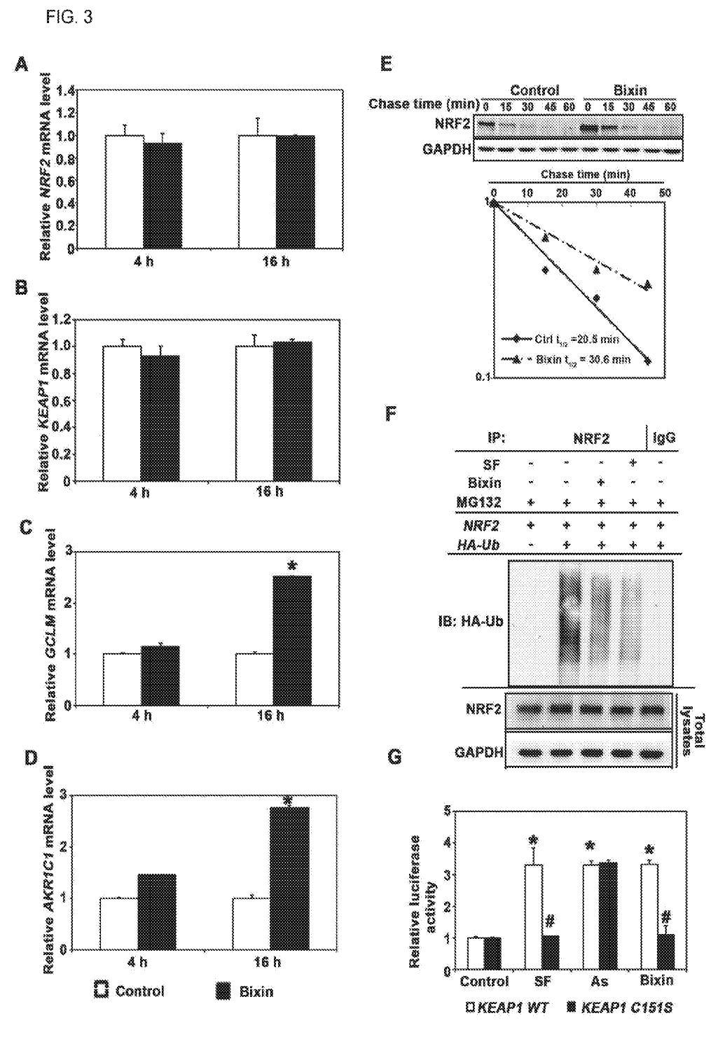

As indicated already by expression array analysis (FIG. 1A), it was also observed that bixin treatment caused pronounced upregulation of NRF2 target genes at the mRNA level without affecting NRF2 or KEAP1 mRNA levels (FIG. 3A-D), suggesting that bixin-based NRF2 activation occurs at the posttranscriptional level [43].

Next, the effect of bixin treatment on the half-life (t_{1/2}) of endogenous NRF2 protein was determined. Cycloheximide was added to untreated or bixin-treated cells to block de novo protein synthesis, and cells were harvested at different time points followed by immunoblot analysis (FIG. 3E, upper panel), and intensity of the NRF2 band was quantified to calculate NRF2 half-life (FIG. 3E, bottom panel). It was observed that t_{1/2} of NRF2 of untreated cells was 20.5 min; however, after bixin treatment t_{1/2} of NRF2 increased to 30.6 min.

In order to test if bixin-based NRF2 activation occurs through inhibition of KEAP1-mediated ubiquitination, a cell-based ubiquitination assay was performed in HaCaT cells cotransfected with expression vectors for NRF2 and HA-tagged ubiquitin (HA-Ub) (FIG. 3F) [43]. To this end, cells were either exposed to bixin (40 μM) or sulforaphane (SF; 5 μM, positive control) or left untreated, combined with proteasome inhibition (MG132; 10 μM, 4 h). In response to bixin exposure, a dramatic reduction of NRF2-ubiquitination compared to the untreated control occurred, a response similar to the established inhibitory effects of SF on NRF2-ubquitination.

Since it has been shown previously that NRF2 activation by canonical inducers (including SF and tBHQ) depends on C (Cys)-151, a critical cysteine residue in KEAP1, the mechanistic involvement of KEAP1-C151 bixin-based NRF2 activation was examined [47, 48]. HaCaT cells were cotransfected with expression vectors for either KEAP1 wild type (KEAP1-WT) or a mutant KEAP1 (KEAP1-C151S; Cys-151 mutated to serine) along with ARE-firefly luciferase and Renilla luciferase reporters to assess NRF2 transcriptional activity (FIG. 3G). After exposure to SF (5 μM), As (sodium arsenite; 5 μM), or bixin (40 μM; all 16 h), NRF2 transcriptional activity was enhanced by all treatments in KEAP1-WT cells, whereas NRF2 activation by SF or bixin was abolished in KEAP1-C151S cells. In contrast, as treatment was equally effective causing NRF2 transcriptional activation in the KEAP1-C151S cells, an observation consistent with a previous report that this compound is a non-canonical NRF2 inducer that operates through a KEAP1-C151-independent mechanism [47, 48]. Taken together, these results demonstrate that bixin is a canonical NRF2 inducer acting through the critical Cys-151 sensor residue in KEAP1.

### Example 3

This example demonstrates that systemic administration of bixin activates cutaneous NRF2 and suppresses UV-induced skin photodamage in Nrf2^{+/+} but not Nrf2^{-/-} mice.

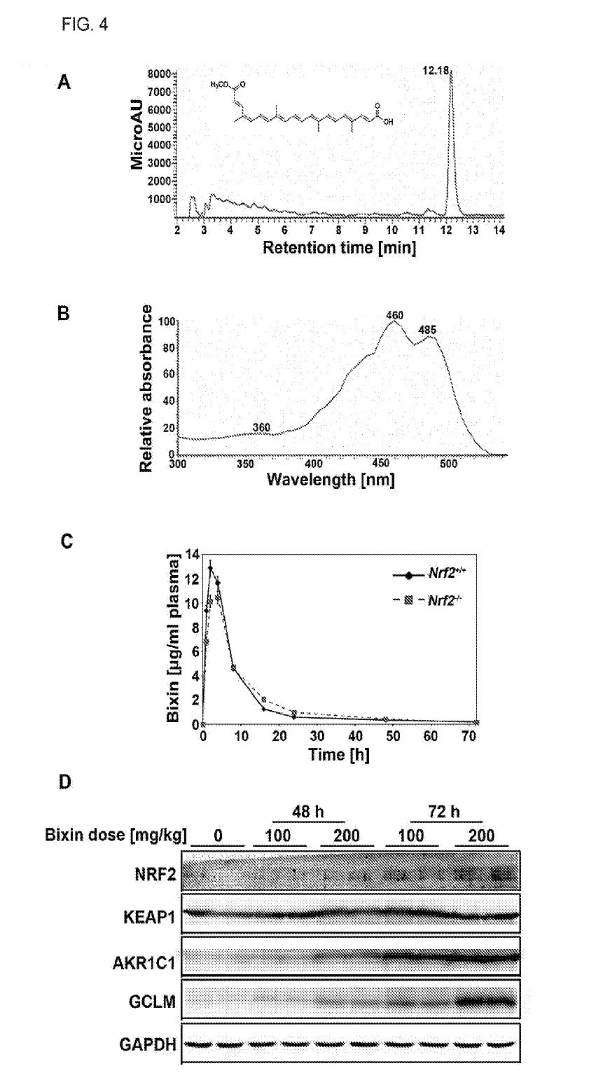

To explore the potential systemic photoprotective activity of bixin in a murine skin sunburn model, the feasibility of upregulating cutaneous NRF2 activity by systemic administration was first examined. It has been reported earlier that efficient cutaneous accumulation of dietary carotenoids requires prolonged nutritional supplementation over weeks and that pharmacokinetic parameters of cutaneous delivery of dietary carotinoids differ between humans and mice [30]. Therefore, in order to circumvent the more complex pharmacokinetics associated with dietary supplementation, an intraperitoneal (i.p.) route for bixin systemic administration followed by examination of cutaneous NRF2 status was chosen. After i.p. injection of bixin (200 mg/kg) performed in Nrf2^{+/+} and Nrf2^{-/-} mice, plasma was collected at 0, 1, 2, 4, 8, 16, 24, 48 and 72 h after injection followed by HPLC-photodiode array detection (FIG. 4A-C). Bixin peak plasma concentrations (Cₘₐₓ; up to 11.3 μg/ml) were reached at 2 h post injection before returning to basal levels at about 48 h, and AUC (‘area under the curve’ equaling total drug exposure) did not differ significantly between genotypes (FIG. 4C). Next, efficacy of various bixin treatment regimens (differing with regard to total dose and time after injection) for maximum activation of the NRF2 pathway in murine skin was determined (FIG. 4D). It was observed that administration of bixin (200 mg/kg, 72 h after injection) was most effective in upregulating cutaneous protein levels of NRF2 and its targets (GCLM and AKR1C1) in Nrf2^{+/+} mice.

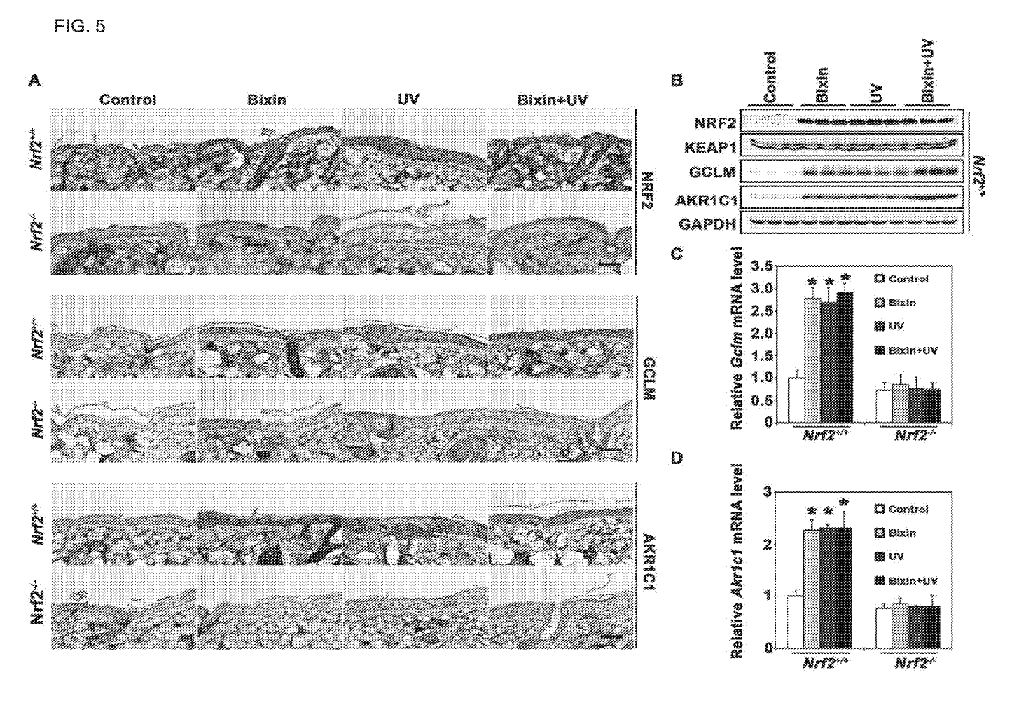

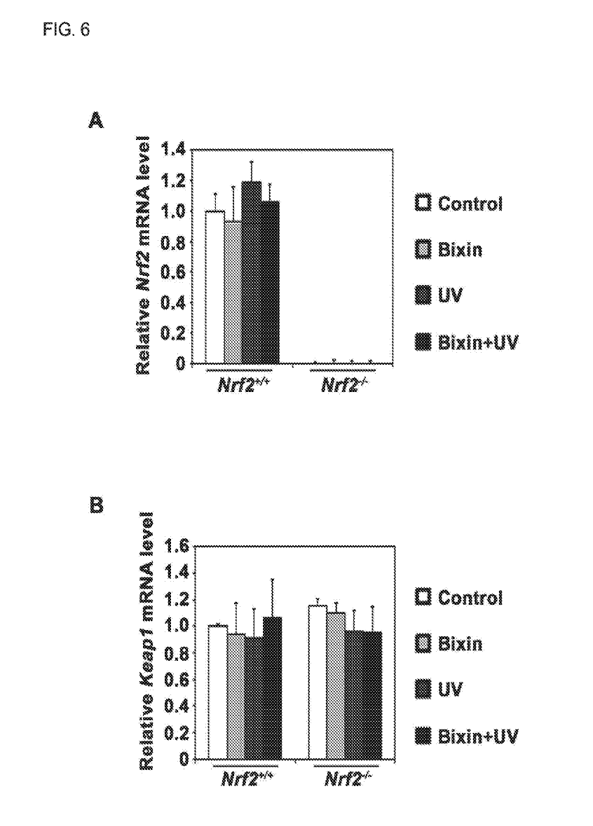

Next, the feasibility of bixin-induced cutaneous protection was studied in a solar UV photodamage model. Nrf2^{+/+} and Nrf2^{-/-} mice were i.p. injected with either corn oil (carrier control) or bixin (200 mg/kg) 48 h before solar UV exposure (240 mJ/cm² UVB; 4.4 J/cm² UVA) [39]. 24 h after UV exposure back skin tissue was then collected followed by immunohistochemical (IHC) analysis. As expected, bixin treatment dramatically induced the cutaneous NRF2 pathway in Nrf2^{+/+} but not in Nrf2^{-/-} mice, detectable at the protein [NRF2, GCLM, AKR1C1 (FIG. 5A-B) and mRNA levels (FIG. 5C-D). The mRNA levels of Nrf2 did not increase in the bixin treatment groups, and bixin had no effects on protein or mRNA levels of Keap1 (FIG. 5B and FIG. 6). Moreover, UV exposure alone activated the NRF2 response in murine skin, effects not detectable in Nrf2^{-/-} mice (FIG. 5).

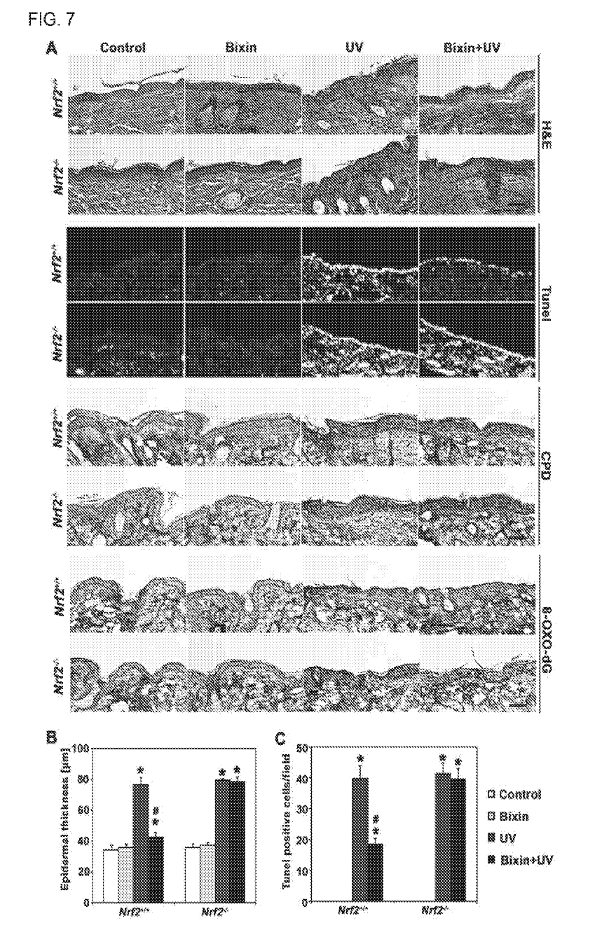

Remarkably, UV exposure caused a pronounced increase in epidermal thickness (FIG. 7A-B), accompanied by the detection of apoptotic (TUNEL-positive) cells located in the basal layer of the epidermis (FIGS. 7A and C), effects that—at the chosen dose level and time point of analysis—occurred irrespective of SKH-1 Nrf2 genotype. In contrast, systemic administration of bixin suppressed UV-induced epidermal thickening and apoptosis, a photoprotective effect confined to Nrf2^{+/+} mice.

### Example 4

This example demonstrates that bixin attenuates solar UV-induced epidermal oxidative DNA damage and inflammatory responses in Nrf2^{+/+} but not in Nrf2^{+/+} mice.

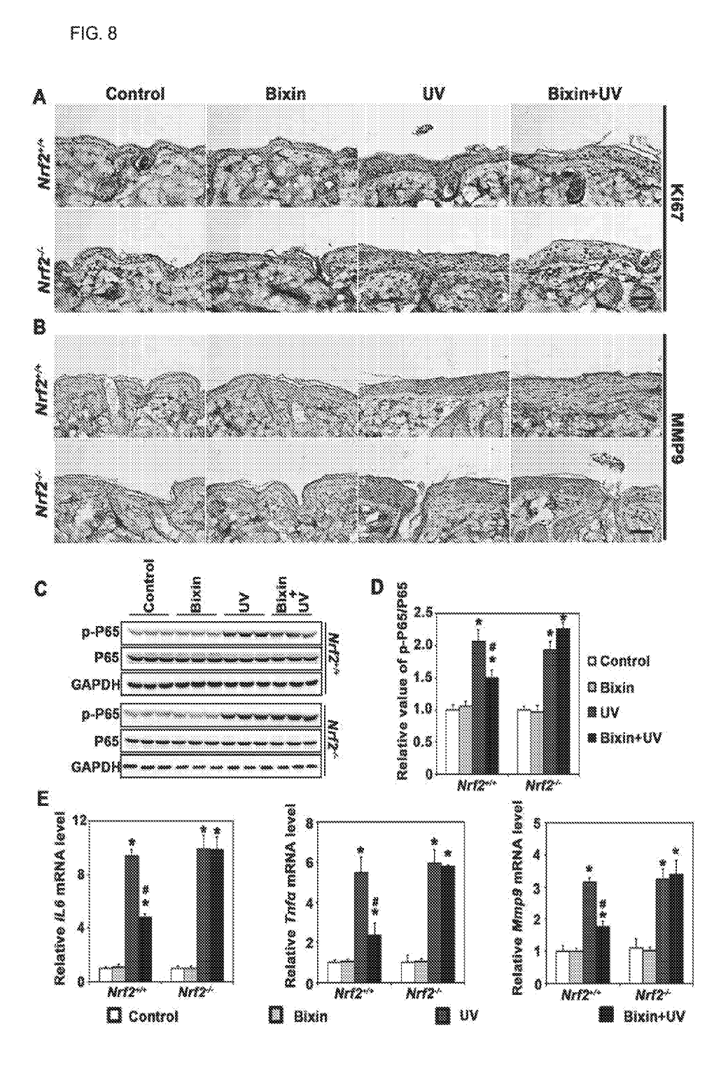

Next, IHC analysis of cutaneous 8-hydroxy-2′-deoxyguanosine (8-oxo-dG), a hallmark of oxidative genomic damage in response to environmental electrophilic insult, revealed that UV-exposure equally enhanced 8-oxo-dG staining in both Nrf2^{+/+} and Nrf2^{-/-} mice, an effect suppressed by bixin administration (FIG. 7A). This photoprotective effect occurred in Nrf2^{+/+} but not Nrf2^{-/-} mice suggesting that bixin-based antioxidant photoprotection is strictly NRF2-dependent. In contrast, cyclobutane pyrimidine dimer (CPD)-lesions, a molecular hallmark of UVB-induced DNA damage that occurs independent of oxidative pathways, was not antagonized by bixin treatment and was equally pronounced in UV-exposed Nrf2^{+/+} and Nrf2^{-/-} mouse skin (FIG. 7A). Consistent with NRF2-dependent suppression of UV-induced epidermal thickening (FIG. 7A-B), the suppression of UV-elicited keratinocyte hyperproliferation in bixin-treated Nrf2^{+/+} mice [as indicated by Ki67 IHC analysis was also observed (FIG. 8A)].

Next, bixin-modulation of UV-induced cutaneous inflammation was examined. IHC analysis revealed that UV-induced epidermal expression of the inflammatory NF-κB target gene MMP9 (matrix metallopeptidase 9; gelatinase B), observable equally in Nrf2^{+/+} and Nrf2^{-/-} mice, was significantly antagonized by systemic delivery of bixin in Nrf2^{+/+} mice only (FIG. 8B). Furthermore, UV-activation of the NF-κB pathway was evident from upregulated p65 phosphorylation (p-p65) detected in both Nrf2^{+/+} and Nrf2^{-/-} mice (FIG. 8C-D). Systemic administration of bixin decreased UV-induced p-p65 accumulation in the skin of Nrf2^{+/+} mice, whereas bixin treatment displayed minimal effects in Nrf2^{-/-} mice, observations consistent with prior reports on Nrf2-dependent attenuation of NF-κB [49].

In order to substantiate the bixin-elicited attenuation of UV-induced activation of the cutaneous NF-κB pathway, modulation of NF-κB target gene expression (IL6, TNFα, MMP9) was monitored at the mRNA level (FIG. 8E). As expected, UV-exposure upregulated cutaneous mRNA levels of NF-κB target genes in both Nrf2^{+/+} and Nrf2^{-/-} mice, an observation consistent with analogous published experiments examining UV-induced upregulation of IL-6, IL-1β, and COX-2 expression in Nrf2^{+/+} and Nrf2^{-/-} SKH-1 mice [9, 22, 49], whereas bixin-attenuation of inflammatory gene expression was observed only in Nrf2^{+/+} mice.

### Example 5

This example provides the materials/methods for Examples 1-4.

### Chemicals, Antibodies, and Cell Culture

Bixin was purchased from Spectrum (New Brunswick, N.J.); sodium arsenite (As), cycloheximide, and MG132 were from Sigma (St. Louis, Mo.); sulforaphane (SF) was purchased from Santa Cruz (Santa Cruz, Calif.); primary antibodies against NRF2, KEAP1, GCLM, AKR1C1, NQO1, AKR1C2, HO-1, TrxR, FTH (heavy), MMP9, Ki67, and GAPDH, as well as horseradish peroxidase (HRP)-conjugated secondary antibodies were purchased from Santa Cruz. Antibodies against p-P65 and P65 were purchased from Cell Signaling. The anti-Thymine Dimer (H3) CPD antibody was purchased from Novus (Littleton, Colo.). The hemagglutinin (HA) epitope antibody was purchased from Covance (Branford, Conn.). The 8-oxo-dG antibody was purchased from Trevigen (Gaithersburg, Md.). Human immortalized HaCaT keratinocytes were grown in Dulbecco's Modified Eagle Medium (DMEM) supplemented with 10% fetal bovine serum and 0.1% gentamycin, and primary human epidermal keratinocytes [adult HEKa (C-005-5C)] were cultured on collagen matrix protein-coated dishes using Epilife medium (EDGS growth supplement; Life Technologies, Carlsbad, Calif.).

Irradiation with Solar UV Light

Irradiation with solar UV occurred as described before [38-40]. A KW large area light source solar simulator, model 91293, from Oriel Corporation (Stratford, Conn.) was used, equipped with a 1000 W Xenon arc lamp power supply, model 68920, and a VIS-IR bandpass blocking filter combined with an atmospheric attenuation filter (output 290-400 nm plus residual 650-800 nm). At 345 mm from the source, the UV dose was 4.4 J/cm² UVA+240 mJ/cm² UVB radiation.

### Bixin Mass Spectrometry and Detection in Mouse Plasma

Electrospray mass spectrometry of bixin [dissolved in tetrahydrofuran and diluted tenfold in acetonitrile/NH₄OH (0.1 N); ESI-MS (negative ion mode) m/z 393.21 (M−1)⁻] was performed using a Bruker Apex FT/ICR mass spectrometer. For determination of bixin plasma levels, mouse samples were subjected to chloroform extraction followed by analysis using a Thermo Finnigan Surveyor HPLC system with photodiode array detector (300-580 nm) using a Luna RP-C18 column (3μ; 100×4.6 mm; Phenomenex, Torrance, Calif.) with mobile phase A (water, 0.1% formic acid) and mobile phase B (acetonitrile, 0.1% formic acid); gradient: 0 min: 20% A; 10 min: 5% A; 15 min 0% A.

### Human Oxidative Stress RT² Profile™ PCR Expression Array Analysis

Total cellular RNA was prepared according to a standard procedure using the RNeasy kit (Qiagen, Valencia, Calif., USA). Reverse transcription was performed using the RT² First Strand kit (SuperArray, Frederick, Md., USA) and 1 μg total RNA. The Human Oxidative Stress RT²Profiler™ PCR Expression Array (SuperArray) profiling the expression of 84 stress-related genes was employed as described before [41, 42]. Gene-specific product was normalized to ACTB and quantified using the comparative (ΔΔCₜ) Ct method following the ABI Prism 7000 sequence detection system user guide. Expression values were averaged across three independent array experiments followed by statistical analysis.

### Cell Viability

Bixin cytotoxicity was assessed by flow cytometric analysis of annexinV/PI-stained cells using a commercial kit from Sigma (APO-AF, St. Louis, Mo.) as published before [40,42]. Bixin toxicity was also assessed examining functional impairment of mitochondria using the 3-(4,5-dimethylthiazol-2-yl)-2,5-diphenyltetrazolium bromide (MTT) standard assay [43. Approximately 1×10⁴ HaCaT cells were seeded in a 96-well plate, and 24 h later the cells were treated with the indicated doses of bixin for another 48 h. After treatment, 20 μL of 2 mg/mL MTT were directly added to the cells, followed by incubation at 37° C. for 2 h. 100 μl of isopropanol/HCl were added to each well and the plate was shaken at room temperature to dissolve the crystals. Absorbance was measured at 570 nm using the Synergy 2 Multi-Mode Microplate Reader (Biotek).

### Glutathione Assay

Total intracellular glutathione in cultured cells was analyzed using the luminescent GSH-Glo glutathione assay (Promega). Cells were harvested and then counted using a Z2 Coulter counter, and GSH was determined per 10,000 viable cells. Data represent relative levels of glutathione normalized for cell number (treated versus solvent controls).

### Detection of Intracellular Oxidative Stress by Flow Cytometric Analysis

Photodynamic induction of intracellular oxidative stress and its suppression by bixin pretreatment was analyzed by flow cytometry using 2′,7′-dichlorodihydrofluorescein diacetate (DCFH-DA) as a sensitive non-fluorescent precursor dye according to a published standard procedure [14]. HaCaT keratinocytes were pretreated with bixin (1 or 24 h) and then exposed to singlet oxygen generated by dye-sensitization as described earlier [14, 44]. In brief, toluidine blue 0 (TB) photosensitization was achieved using a ‘Sylvania 15 W Cool White’ light tube delivering visible light at an irradiance of 4.29 mW/cm². The irradiance in the visible region (400-700 nm) was determined using a spectroradiometer, Model 754 from Optronic Laboratories (Orlando, Fla.). Cells received visible radiation at a distance of 50 mm from the source through the polystyrene lids of cell culture dishes. For ¹O₂ exposure, cells were washed with PBS and immediately exposed to the combined action of visible light (0.3 J/cm²) and TB (3.3 μM) in PBS. Following 5 min incubation in the dark after irradiation, cells were washed with PBS. Cells were then incubated for 60 min in the dark (37° C., 5% CO₂) with culture medium containing DCFH-DA (5 μg/mL final concentration). Cells were then washed with PBS, harvested by trypsinization, resuspended in 300 μl PBS, and analyzed by flow cytometry.

Transfection of cDNA and Luciferase Reporter Gene Assay

Transfection of cDNA was performed using Lipofectamine 3000 (Invitrogen) according to the manufacturer's instructions. Activation of NRF2 transcriptional activity was performed as previously published [14, 43]. Briefly, HaCaT cells were cotransfected with expression vectors for either KEAP1 wild type (KEAP1-WT) or KEAP1 with a mutation that generates a protein that contains a serine residue instead of a cysteine (KEAP1-C151S), along with NQO1-ARE firefly luciferase and Renilla luciferase reporters. At 24 h post-transfection, cells were left untreated or treated with SF (5 μM), As (5 μM), or bixin (40 μM) for 16 h. The cells were then lysed for analysis of the reporter gene activity using the Promega dual-luciferase reporter gene assay system.

### Immunoblot Analysis, Ubiquitylation Assay, and Protein Half-Life

Experiments were performed according to previously published procedures [43]. Cells were harvested in sample buffer (50 mM Tris-HCl [pH 6.8], 2% sodium dodecyl sulfate (SDS), 10% glycerol, 100 mM dithiothreitol (DTT), and 0.1% bromophenol blue), boiled and sonicated. Total cell lysates were resolved by SDS-PAGE and subjected to immunoblot analyses with the indicated antibodies. For the ubiquitination assay, cells were cotransfected with expression vectors for NRF2 and HA-tagged ubiquitin (HA-Ub). Cells were left untreated or treated with either 5 μM sulforaphane (SF) or 40 μM bixin along with 10 μM MG132 for 4 h. Cells were harvested in buffer containing 2% SDS, 150 mM NaCl, 10 mM Tris-HCl (pH 8.0), and 1 mM DTT and boiled immediately. For immunoprecipitation, 1 μg of NRF2 antibody was incubated with the cell lysates at 4° C. overnight with protein A agarose beads (Invitrogen). Immunoprecipitated complexes were washed four times with RIPA buffer and eluted in sample buffer by boiling for 5 min. Samples were resolved by SDS-PAGE and immunoblotted with HA antibody. To measure the half-life of NRF2, HaCaT cells were either left untreated or treated with 5 μM bixin for 4 h, then 50 μM cycloheximide was added to block protein synthesis. Total cell lysates were collected at different time points and subjected to immunoblot analysis with NRF2 antibody. The relative intensity of the bands was quantified using the ChemiDoc CRS gel documentation system and Quantity One software (BioRad).

mRNA Extraction and Real-Time RT-PCR

Total RNA was extracted from HaCaT cells and mouse skin tissues using TRIzol (Invitrogen). Equal amounts of mRNA were used to generate cDNA using the M-MLV Reverse Transcriptase synthesis kit according to the manufacturer's instructions (Promega). RT-PCR and primer sequences of NRF2, KEAP1, GCLM, AKR1C1 and GAPDH were described previously to evaluate mRNA expression using the LightCycler 480 system (Roche) [21, 43]. Quantification of cDNA amount for mouse Nrf2, Keap1, Gclm, and Akr1c1 in each skin tissue sample was performed using the KAPA SYBR FAST qPCR Kit (Kapa Biosystems).

All primer sets were designed with Primer 3 online free software (http://www-genome.wi.mit.edu/genome_software/other/primer3.html) and were synthesized by Sigma as follows:

Nrf2:
forward
(SEQ ID NO: 1)
(CTCAGCATGATGGACTTGGA)
reverse
(SEQ ID NO: 2)
(TCTTGCCTCCAAAGGATGTC);
Keap1:
forward
(SEQ ID NO: 3)
(GATCGGCTGCACTGAACTG)
reverse
(SEQ ID NO: 4)
(GGCAGTGTGACAGGTTGAAG);
Akr1c1:
forward
(SEQ ID NO: 5)
(GGAGGCCATGGAGAAGTGTA)
reverse
(SEQ ID NO: 6)
(GCACACAGGCTTGTACCTGA);
Gclm:
forward
(SEQ ID NO: 7)
(TCCCATGCAGTGGAGAAGAT)
reverse
(SEQ ID NO: 8)
(AGCTGTGCAACTCCAAGGAC);
IL6:
forward
(SEQ ID NO: 9)
(CCGGAGAGGAGACTTCACAG)
reverse
(SEQ ID NO: 10)
(TCCACGATTTCCCAGAGAAC);
TNFα:
forward
(SEQ ID NO: 11)
(AGCCCCCAGTCTGTATCCTT)
reverse
(SEQ ID NO: 12)
(GGTCACTGTCCCAGCATCTT);
Mmp9:
forward
(SEQ ID NO: 13)
(CAATCCTTGCAATGTGGATG)
reverse
(SEQ ID NO: 14)
(AGTAAGGAAGGGGCCCTGTA;
β-actin:
forward
(SEQ ID NO: 15)
(AAGGCCAACCGTGAAAAGAT)
reverse
(SEQ ID NO: 16)
(GTGGTACGACCAGAGGCATAC).

The RT-PCR conditions used were the following: one cycle of initial denaturation (95° C. for 3 min), 40 cycles of amplification (95° C. for 10 s, 60° C. for 20 s, and 72° C. for 5 s), melting curve (95° C. for 5 s, 65° C. for 1 min, and 97° C. continuous), and a cooling cycle (40° C. for 30 s). Mean crossing point (Cp) values and standard deviations (SD) were determined. Cp values were normalized to the respective Cp values of the mouse β-actin reference gene. Data are presented as a fold change in gene expression compared to the control group.

### Animals and Treatments

Nrf2^{+/+} and Nrf2^{-/-} SKH-I mice were obtained by breeding Nrf2 heterozygous mice generated by back-crossing Nrf2^{-/-} C57BL/6 mice onto the SKH-1 hairless mouse genetic background for at least six generations (JAX® Mice, The Jackson Laboratory) [45]. All animals received water and food ad libitum and were handled according to the Guide for the Care and Use of Laboratory Animals; the protocols were approved by the University of Arizona Institutional Animal Care and Use Committee. Eight-week-old Nrf2^{+/+} and Nrf2^{-/-} mice were randomly allocated into four groups (n=6): (i) control (corn oil); (ii) bixin (200 mg/kg, dissolved in corn oil); (iii) UV; (iv) bixin+UV. Bixin was administered through intraperitoneal (i.p.) injection 48 h before UV exposure.

### Skin Tissue Collection, H&E Staining, and IHC

24 h after UV exposure, the mice were euthanized and back skin was collected and divided into two parts: one part was frozen in liquid nitrogen for total RNA extraction and protein analysis; the other part was fixed in 10% buffered formalin and embedded in paraffin for histological and immunohistochemical analyses. Tissue sections (4 μm) were baked and deparaffinized. Hematoxylin and eosin (H&E) staining was performed for pathological examination. Antigen retrieval was carried out by boiling the slides with retrieval solution (citric acid monohydrate 2.1 g/L in H₂O pH=6.0) three times for 5 min [43]. Tissue sections were then exposed to 3.5 M HCl for 15 min at room temperature and washed with PBS. Subsequently, tissue sections were treated with 0.3% peroxidase to quench endogenous peroxidase activity. Tissue sections were incubated with 5% normal goat serum for 30 min followed by 2 h incubation with primary antibodies at 1:100 dilution at room temperature. Staining was performed using the EnVision+System-HRP (DAB) kit (Dako) according to the manufacturer's instructions.

### In Situ TUNEL Assay

An in situ cell death detection kit (Roche) was used for detection of apoptotic cell death in skin sections according to the manufacturer's instructions. Briefly, tissue sections were pretreated with proteinase K (20 μg/ml) in 10 mM Tris/HCl (pH 7.8) at 37° C. for 30 min. After washing three times with PBS, tissue sections were incubated with TUNEL reaction mixture for 1 h at 37° C. in the dark. Tissue sections were then stained with Hoechst, and analyzed using a fluorescence microscope (Zeiss Observer.Zi microscope; slidebook computer program; excitation wavelength: 450-500 nm; detection wavelength: 515-565 nm). Hoechst stain was visualized under UV light.

### Statistical Analysis

Results are presented as the mean±SD of at least three independent experiments performed in duplicate or triplicate each. Statistical tests were performed using SPSS 13.0. Unpaired Student's t-tests were used to compare the means of two groups, and selected data sets were analyzed employing one-way analysis of variance (ANOVA) with Tukey's post hoc test; differences between groups were considered significant at p<0.05.

### Example 6

This example demonstrates that administration of 1% Bixin in topical carrier activates Nrf2 and Nrf2 target expression in skin tissues of SKH-1 mice.

Experiments were conducted that indicate feasibility of topical skin administration of one of the prototype Nrf2 inducer compounds causing the activation of cytoprotective gene expression. Results indicate that activation of Nrf2 and Nrf2 target expression in skin tissues of SKH-1 mice is dependent on specific topical formulation.

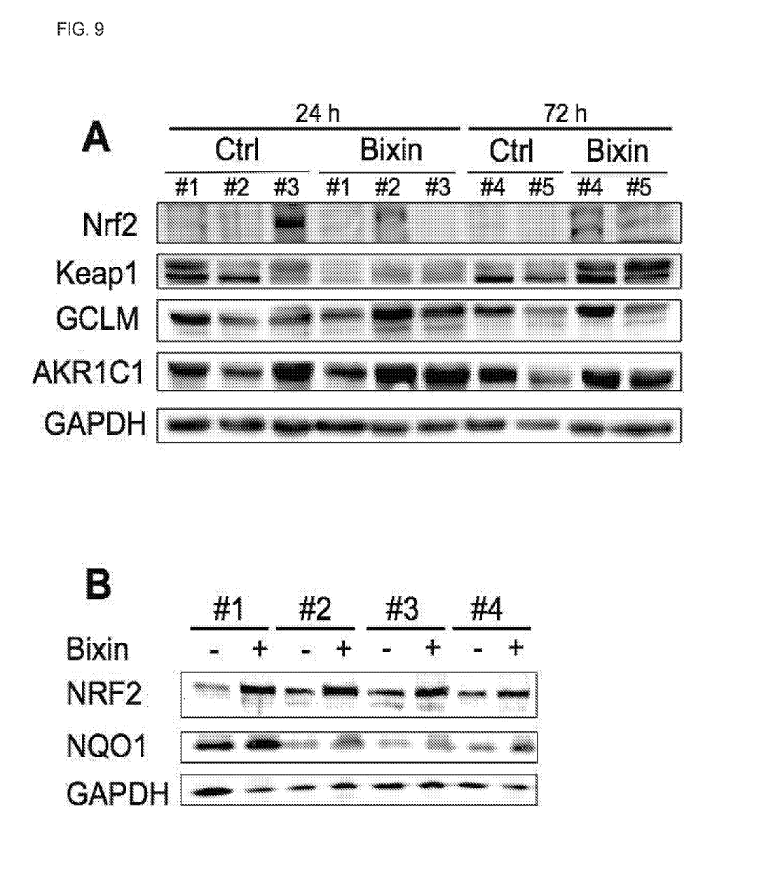

FIG. 9A shows 1% Bixin in a standard topical carrier (Vanicream) is not efficient in upregulating cutaneous Nrf2. SKH-1 mice were treated with topical Bixin (1% in Vanicream carrier; 50 μl per application) (‘Bixin’). As a control, skin at a different anatomical location was also treated with carrier only (Ctrl). After 24 h (mice #1, #2, #3) or 72 h (mice #4, #5) were sacrificed and skin was harvested for immunoblot analysis testing for Nrf2 upregulation (Nrf2/Keap1) and Nrf2 target gene (GCLM, AKR1C1) expression. GAPDH served as loading control. Topical Bixin in vanicream was shown to not cause a reproducible and significant activation of Nrf2 and Nrf2 target gene expression.

In contrast, FIG. 9B shows that 1% Bixin in PEG400 (e.g., polyethylene glycol; average mass 400 Da) is very efficient in upregulating the Nrf2-dependent cytoprotective response with topical administration. SKH-1 mice were treated with topical Bixin (1% in PEG400; Poly(ethylene glycol) average Mₙ 400; Number Average Molecular Weight, Mn; from SigmaAldrich Chemicals) (‘Bixin: +’; 50 μl per application). As a control, skin at a different anatomical location was also treated with PEG400 carrier only (‘Bixin: −’). After 24 h (mice #1, #2, #3, #4) were sacrificed and skin was harvested for immunoblot analysis testing for Nrf2 upregulation (Nrf2) and Nrf2 target gene (NQO1) expression. GAPDH served as loading control. Topical Bixin in PEG400 was shown to cause a reproducible and significant activation of Nrf2 and Nrf2 target gene expression.

### Example 7

This example demonstrates that Bixin treatment induces Nrf2 and Nrf2 target gene expression in human primary skin melanocytes.

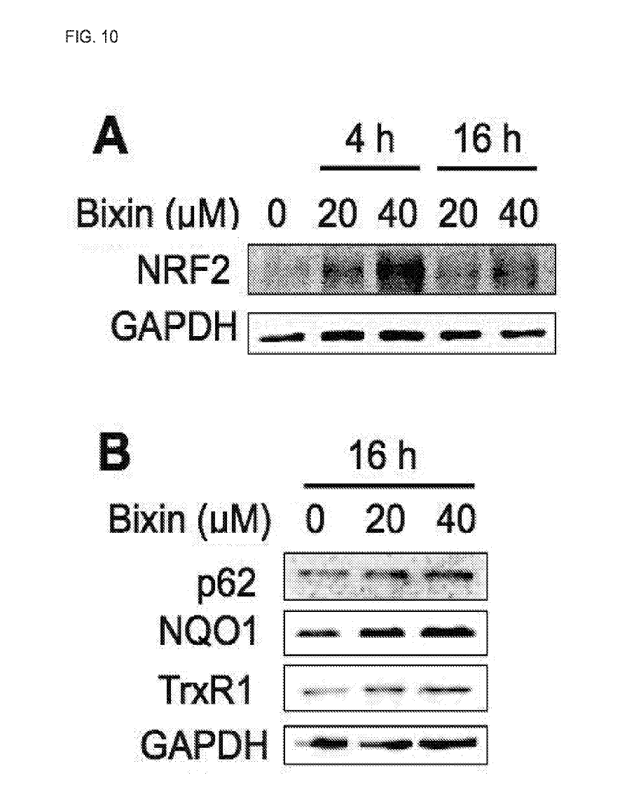

Human cultured primary melanocytes (skin pigment producing cells) were treated with Bixin (0-40 μM) for up to 16 h. After 4 and 16 h, cells were harvested for immunoblot analysis testing for Nrf2 upregulation (Nrf2) GAPDH served as loading control. FIG. 10A shows that Bixin is an efficient Nrf2 activator in human skin melanocytes.

Human cultured primary melanocytes (skin pigment producing cells) were treated with Bixin (0-40 μM) for 16 h. After 16 h, cells were harvested for immunoblot analysis testing for upregulation of cytoprotective Nrf2 targets including NQO1 and TrxR1. GAPDH served as loading control. FIG. 10B shows that Bixin is an efficient activator of cytoprotective Nrf2 target gene expression in human skin melanocytes.

### Example 8

This example demonstrates that irradiation of bixin with solar ultraviolet light enhances (‘potentiates’) bixin activity for upregulation of cytoprotective gene expression in human skin keratinocytes.

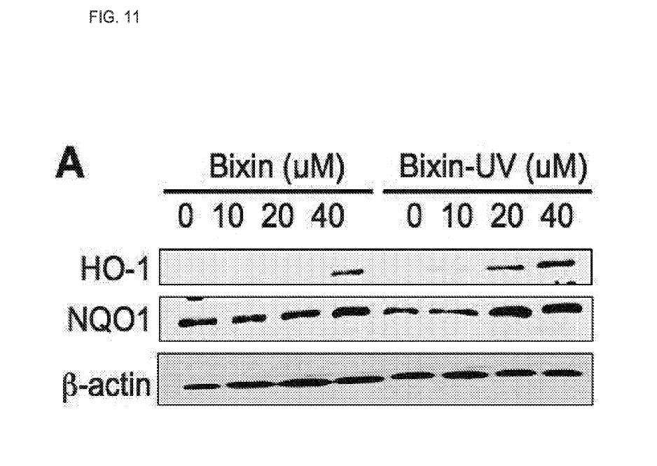

Human cultured skin keratinocytes (HaCaT) were treated with Bixin (0-40 μM) for up 16 h. For comparison, bixin was exposed to solar ultraviolet radiation and then used to treat cells (‘Bixin-UV’; UVA: 23 J/cm²; UVB: 1200 mJ/cm²; ‘Bixin-UV). After 16 h, cells were harvested for immunoblot analysis testing for upregulation of cytoprotective Nrf2 target genes: HO-1 and NQO1. β-Actin served as a loading control. FIG. 11 shows that UV-exposure of Bixin enhances bixin potency as an activator of Nrf2 target gene expression in human skin keratinocytes.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes. The following references are referenced within this application and are herein incorporated by reference in all entireties:
[1] Chen, H.; Weng, Q. V., Fisher, D. E., UV signaling pathways within the skin, J. Invest. Dermatol., 134:2080-2085; 2014.
[2] Natarajan, V. T.; Ganju, P.; Ramkumar, A.; Grover, R., Gokhale, R. S., Multifaceted pathways protect human skin from UV radiation, Nat. Chem. Biol., 10:542-551; 2014.
[3] Brash, D. E., UV signature mutations, Photochem. Photobiol., 91:15-26; 2015.
[4] Gonzalez, S.; Astner, S.; An, W.; Goukassian, D., Pathak, M. A., Dietary lutein/zeaxanthin decreases ultraviolet B-induced epidermal hyperproliferation and acute inflammation in hairless mice, J. Invest. Dermatol., 121:399-405; 2003.
[5] Wondrak, G T.; Jacobson, M. K., Jacobson, E. L., Endogenous UVA-photosensitizers: mediators of skin photodamage and novel targets for skin photoprotection, Photochem. Photobiol. Sci., 5:215-237; 2006.
[6] Nichols, J. A., Katiyar, S. K., Skin photoprotection by natural polyphenols: anti-inflammatory, antioxidant and DNA repair mechanisms, Arch. Dermatol. Res., 302:71-83; 2010.
[7] Wondrak, G T., Sunscreen-Based Skin Protection Against Solar Insult: Molecular Mechanisms and Opportunities, in: D. Alberts (Ed.) Fundamentals of Cancer Prevention, Springer Science & Business Media2014, pp. 301-320.
[8] Suzuki, T.; Motohashi, H., Yamamoto, M., Toward clinical application of the Keap1-Nrf2 pathway, Trends Pharmacol. Sci., 34:340-346; 2013.
[9] Saw, C. L.; Huang, M. T.; Liu, Y.; Khor, T. O.; Conney, A. H., Kong, A. N., Impact of Nrf2 on UVB-induced skin inflammation/photoprotection and photoprotective effect of sulforaphane, Mol. Carcinog., 50:479-486; 2011.
[10] Ma, Q., Role of Nrf2 in oxidative stress and toxicity, Annu. Rev. Pharmacol. Toxicol., 53:401-426; 2013.
[11] Schafer, M., Werner, S., Nrf2-A regulator of keratinocyte redox signaling, Free Radic. Biol. Med., 2015 Apr. 23; [Epub ahead of print].
[12] Hirota, A.; Kawachi, Y.; Itoh, K.; Nakamura, Y.; Xu, X.; Banno, T.; Takahashi, T.; Yamamoto, M., Otsuka, F., Ultraviolet A irradiation induces NF-E2-related factor 2 activation in dermal fibroblasts: protective role in UVA-induced apoptosis, J. Invest. Dermatol., 124:825-832; 2005.
[13] Dinkova-Kostova, A. T.; Jenkins, S. N.; Fahey, J. W.; Ye, L.; Wehage, S. L.; Liby, K. T.; Stephenson, K. K.; Wade, K. L., Talalay, P., Protection against UV-light-induced skin carcinogenesis in SKH-1 high-risk mice by sulforaphane-containing broccoli sprout extracts, Cancer Lett., 240:243-252; 2006.
[14] Wondrak, G T.; Cabello, C. M.; Villeneuve, N. F.; Zhang, S.; Ley, S.; Li, Y.; Sun, Z., Zhang, D. D., Cinnamoyl-based Nrf2-activators targeting human skin cell photo-oxidative stress, Free Radic. Biol. Med., 45:385-395; 2008.
[15] Benedict, A. L.; Knatko, E. V., Dinkova-Kostova, A. T., The indirect antioxidant sulforaphane protects against thiopurine-mediated photooxidative stress, Carcinogenesis, 33:2457-2466; 2012.
[16] Gruber, F.; Mayer, H.; Lengauer, B.; Mlitz, V.; Sanders, J. M.; Kadl, A.; Bilban, M.; de Martin, R.; Wagner, 0.; Kensler, T. W.; Yamamoto, M.; Leitinger, N., Tschachler, E., NF-E2-related factor 2 regulates the stress response to UVA-1-oxidized phospholipids in skin cells, FASEB J., 24:39-48; 2010.
[17] Tian, F. F.; Zhang, F. F.; Lai, X. D.; Wang, L. J.; Yang, L.; Wang, X.; Singh, G, Zhong, J. L., Nrf2-mediated protection against UVA radiation in human skin keratinocytes, Biosci. Trends, 5:23-29; 2011.
[18] Hirota, A.; Kawachi, Y.; Yamamoto, M.; Koga, T.; Hamada, K., Otsuka, F., Acceleration of UVB-induced photoageing in nrf2 gene-deficient mice, Exp. Dermatol., 20:664-668; 2011.
[19] Kalra, S.; Knatko, E. V.; Zhang, Y.; Honda, T.; Yamamoto, M., Dinkova-Kostova, A. T., Highly potent activation of Nrf2 by topical tricyclic bis(cyano enone): implications for protection against UV radiation during thiopurine therapy, Cancer Prev. Res. (Phila), 5:973-981; 2012.
[20] Schafer, M.; Dutsch, S.; auf dem Keller, U.; Navid, F.; Schwarz, A.; Johnson, D. A.; Johnson, J. A., Werner, S., Nrf2 establishes a glutathione-mediated gradient of UVB cytoprotection in the epidermis, Genes Dev., 24:1045-1058; 2010.
[21] Tao, S.; Justiniano, R.; Zhang, D. D., Wondrak, G T., The Nrf2-inducers tanshinone I and dihydrotanshinone protect human skin cells and reconstructed human skin against solar simulated UV, Redox Biol., 1:532-541; 2013.
[22] Knatko, E. V.; Ibbotson, S. H.; Zhang, Y.; Higgins, M.; Fahey, J. W.; Talalay, P.; Dawa, R.; Ferguson, J.; Huang, J. T.; Clarke, R.; Zheng, S.; Saito, A.; Kalra, S.; Benedict, A. L.; Honda, T.; Proby, C. M., Dinkova-Kostova, A. T., Nrf2 activation protects against solar-simulated ultraviolet radiation in mice and humans, Cancer Prev. Res. (Phila), 8: 475-86; 2015.
[23] Chun, K. S.; Kundu, J.; Kundu, J. K., Surh, Y. J., Targeting Nrf2-Keap1 signaling for chemoprevention of skin carcinogenesis with bioactive phytochemicals, Toxicol. Lett., 229:73-84; 2014.
[24] Lieder, F.; Reisen, F.; Geppert, T.; Sollberger, G; Beer, H. D.; auf dem Keller, U.; Schafer, M.; Detmar, M.; Schneider, G, Werner, S., Identification of UV-protective activators of nuclear factor erythroid-derived 2-related factor 2 (Nrf2) by combining a chemical library screen with computer-based virtual screening, J Biol. Chem., 287:33001-33013; 2012.
[25] Schafer, M.; Farwanah, H.; Willrodt, A. H.; Huebner, A. J.; Sandhoff, K.; Roop, D.; Hohl, D.; Bloch, W., Werner, S., Nrf2 links epidermal barrier function with antioxidant defense, EMBO Mol. Med., 4:364-379; 2012.
[26] Dinkova-Kostova, A. T.; Fahey, J. W.; Benedict, A. L.; Jenkins, S. N.; Ye, L.; Wehage, S. L., Talalay, P., Dietary glucoraphanin-rich broccoli sprout extracts protect against UV radiation-induced skin carcinogenesis in SKH-1 hairless mice, Photochem. Photobiol. Sci., 9:597-600; 2010.
[27] Sies, H., Stahl, W., Nutritional Protection Against Skin Damage From Sunlight, Annu. Rev. Nutr., 24:173-200; 2004.
[28] Astner, S.; Wu, A.; Chen, J.; Philips, N.; Rius-Diaz, F.; Parrado, C.; Mihm, M. C.; Goukassian, D. A.; Pathak, M. A., Gonzalez, S., Dietary lutein/zeaxanthin partially reduces photoaging and photocarcinogenesis in chronically UVB-irradiated Skh-1 hairless mice, Skin Pharmacol. Physiol., 20:283-291; 2007.
[29] Stahl, W., Sies, H., beta-Carotene and other carotenoids in protection from sunlight, Am. J. Clin. Nutr., 96:1179S-1184S; 2012.
[30] Fernandez-Garcia, E., Skin protection against UV light by dietary antioxidants, Food Funct., 5:1994-2003; 2014.
[31] Di Mascio, P.; Kaiser, S., Sies, H., Lycopene as the most efficient biological carotenoid singlet oxygen quencher, Arch. Biochem. Biophys., 274:532-538; 1989.
[32] Di Mascio, P.; Devasagayam, T. P.; Kaiser, S., Sies, H., Carotenoids, tocopherols and thiols as biological singlet molecular oxygen quenchers, Biochem. Soc. Trans., 18:1054-1056; 1990.
[33] Ulbricht, C.; Windsor, R. C.; Brigham, A.; Bryan, J. K.; Conquer, J.; Costa, D.; Giese, N.; Guilford, J.; Higdon, E. R.; Holmes, K.; Isaac, R.; Jingst, S.; Kats, J.; Peery, L.; Rusie, E.; Savinainen, A.; Schoen, T.; Stock, T.; Tanguay-Colucci, S., Weissner, W., An evidence-based systematic review of annatto (Bixa orellana L.) by the Natural Standard Research Collaboration, J. Diet Suppl., 9:57-77; 2012.
[34] Stohs, S. J., Safety and efficacy of Bixa orellana (achiote, annatto) leaf extracts, Phytother. Res., 28:956-960; 2014.
[35] Levy, L. W.; Regalado, E.; Navarrete, S., Watkins, R. H., Bixin and norbixin in human plasma: determination and study of the absorption of a single dose of Annatto food color, Analyst, 122:977-980; 1997.
[36] Junior, A. C.; Asad, L. M.; Oliveira, E. B.; Kovary, K.; Asad, N. R., Felzenszwalb, I., Antigenotoxic and antimutagenic potential of an annatto pigment (norbixin) against oxidative stress, Genet. Mol. Res., 4:94-99; 2005.
[37] World Health, O., Evaluation of certain food additives and contaminants, World Health Organ. Tech. Rep. Ser., 1-75, back cover; 2013.
[38] Wondrak, G T.; Roberts, M. J.; Cervantes-Laurean, D.; Jacobson, M. K., Jacobson, E. L., Proteins of the Extracellular Matrix Are Sensitizers of Photo-oxidative Stress in Human Skin Cells, J. Invest. Dermatol., 121:578-586; 2003.
[39] Williams, J. D.; Bermudez, Y.; Park, S. L.; Stratton, S. P.; Uchida, K.; Hurst, C. A., Wondrak, G T., Malondialdehyde-derived epitopes in human skin result from acute exposure to solar UV and occur in nonmelanoma skin cancer tissue, J. Photochem. Photobiol. B, 132:56-65; 2014.
[40] Park, S. L.; Justiniano, R.; Williams, J. D.; Cabello, C. M.; Qiao, S., Wondrak, G T., The Tryptophan-Derived Endogenous Aryl Hydrocarbon Receptor Ligand 6-Formylindolo[3,2-b]Carbazole Is a Nanomolar UVA Photosensitizer in Epidermal Keratinocytes, J. Invest. Dermatol., 135:1649-1658; 2015.
[41] Lamore, S. D.; Qiao, S.; Horn, D., Wondrak, G T., Proteomic identification of cathepsin B and nucleophosmin as novel UVA-targets in human skin fibroblasts, Photochem. Photobiol., 86:1307-1317; 2010.
[42] Qiao, S.; Tao, S.; Rojo de la Vega, M.; Park, S. L.; Vonderfecht, A. A.; Jacobs, S. L.; Zhang, D. D., Wondrak, G T., The antimalarial amodiaquine causes autophagic-lysosomal and proliferative blockade sensitizing human melanoma cells to starvation- and chemotherapy-induced cell death, Autophagy, 9:2087-2102; 2013.
[43] Tao, S.; Zheng, Y.; Lau, A.; Jaramillo, M. C.; Chau, B. T.; Lantz, R. C.; Wong, P. K.; Wondrak, GT., Zhang, D. D., Tanshinone I Activates the Nrf2-Dependent Antioxidant Response and Protects Against As(III)-Induced Lung Inflammation In Vitro and In Vivo, Antioxid. Redox Signal., 19:1647-61; 2013.
[44] Wondrak, G T.; Jacobson, M. K., Jacobson, E. L., Identification of quenchers of photoexcited states as novel agents for skin photoprotection, J. Pharmacol. Exp. Ther., 312:482-491; 2005.
[45] Moi, P.; Chan, K.; Asunis, I.; Cao, A., Kan, Y. W., Isolation of NF-E2-related factor 2 (Nrf2), a NF-E2-like basic leucine zipper transcriptional activator that binds to the tandem NF-E2/AP1 repeat of the beta-globin locus control region, Proc. Natl. Acad. Sci. USA, 91:9926-9930; 1994.
[46] Monticone, M.; Taherian, R.; Stigliani, S.; Carra, E.; Monteghirfo, S.; Longo, L.; Daga, A.; Dono, M.; Zupo, S.; Giaretti, W., Castagnola, P., NAC, tiron and trolox impair survival of cell cultures containing glioblastoma tumorigenic initiating cells by inhibition of cell cycle progression, PLoS One, 9:e90085; 2014.
[47] Zhang, D. D., Hannink, M., Distinct cysteine residues in Keap1 are required for Keap1-dependent ubiquitination of Nrf2 and for stabilization of Nrf2 by chemopreventive agents and oxidative stress, Mol. Cell. Biol., 23:8137-8151; 2003.
[48] Lau, A.; Zheng, Y.; Tao, S.; Wang, H.; Whitman, S. A.; White, E., Zhang, D. D., Arsenic Inhibits Autophagic Flux, Activating the Nrf2-Keap1 Pathway in a p62-Dependent Manner, Mol. Cell. Biol., 33:2436-2446; 2013.
[49] Li, W.; Khor, T. O.; Xu, C.; Shen, G; Jeong, W. S.; Yu, S., Kong, A. N., Activation of Nrf2-antioxidant signaling attenuates NFkappaB-inflammatory response and elicits apoptosis, Biochem. Pharmacol., 76:1485-1489; 2008.
[50] Kim, YR.; Oh, J. E.; Kim, M. S.; Kang, M. R.; Park, S. W.; Han, J. Y.; Eom, H. S.; Yoo, N. J., Lee, S. H., Oncogenic NRF2 mutations in squamous cell carcinomas of oesophagus and skin, J. Pathol., 220:446-451; 2010.
[51] Schafer, M.; Willrodt, A. H.; Kurinna, S.; Link, A. S.; Farwanah, H.; Geusau, A.; Gruber, F.; Sorg, O.; Huebner, A. J.; Roop, D. R.; Sandhoff, K.; Saurat, J. H.; Tschachler, E.; Schneider, M. R.; Langbein, L.; Bloch, W.; Beer, H. D., Werner, S., Activation of Nrf2 in keratinocytes causes chloracne (MADISH)-like skin disease in mice, EMBO Mol. Med., 6:442-457; 2014.
[52] Vayalil, P. K.; Mittal, A.; Hara, Y.; Elmets, C. A., Katiyar, S. K., Green tea polyphenols prevent ultraviolet light-induced oxidative damage and matrix metalloproteinases expression in mouse skin, J. Invest. Dermatol., 122:1480-1487; 2004.
[53] Gonzalez, S.; Gilaberte, Y, Philips, N., Mechanistic insights in the use of a Polypodium leucotomos extract as an oral and topical photoprotective agent, Photochem. Photobiol. Sci., 9:559-563; 2010.
[54] Chen, A. C.; Damian, D. L., Halliday, G M., Oral and systemic photoprotection, Photodermatol. Photoimmunol. Photomed., 30:102-111; 2014.
[55] Somacal, S.; Figueiredo, C. G; Quatrin, A.; Ruviaro, A. R.; Conte, L.; Augusti, P. R.; Roehrs, M.; Denardin, I. T.; Kasten, J.; da Veiga, M. L.; Duarte, M. M., Emanuelli, T., The antiatherogenic effect of bixin in hypercholesterolemic rabbits is associated to the improvement of lipid profile and to its antioxidant and anti-inflammatory effects, Mol. Cell. Biochem., 403:243-253; 2015.
[56] Roehrs, M.; Figueiredo, C. G; Zanchi, M. M.; Bochi, G V.; Moresco, R. N.; Quatrin, A.; Somacal, S.; Conte, L., Emanuelli, T., Bixin and norbixin have opposite effects on glycemia, lipidemia, and oxidative stress in streptozotocin-induced diabetic rats, Int. J. Endocrinol., 2014:839095; 2014.
[57] WHO, Evaluation of certain food additives and contaminants. Thirty-fifth report of the Joint FAO/WHO Expert Committee on Food Additives., Tech. Rep. Ser. 789:1-48; 1990.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A composition comprising a compound having a structure similar to 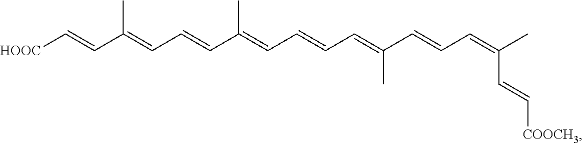 [SMILES: COC(=O)/C=C/C(C)=C\C=C\C(C)=C\C=C\C=C(C)\C=C\C=C(C)\C=C\C(=O)O]
including pharmaceutically acceptable salts, solvates, and/or prodrugs thereof, wherein said compound is capable of activating NRF2 pathway related activity.

2. The composition of claim 1, wherein the compound is capable of activating NRF2 pathway related activity through interaction with KEAP1 wherein interaction with KEAP1 occurs via Cys151 of KEAP1.

3. (canceled)

4. The composition of claim 1, wherein the composition further comprises polyethylene glycol, wherein the amount of the compound within the composition comprising the compound and polyethylene glycol is approximately 1%.

5. (canceled)

6. The composition of claim 1,
wherein said compound is capable of activating NRF2 pathway related activity in keratinocyte cells, and/or
wherein said compound is capable of activating NRF2 pathway related activity in pigment cells, wherein the pigment cells are melanocyte cells.

7. (canceled)

8. (canceled)

9. The composition of claim 1, wherein the compound is irradiated, wherein the source of irradiation is selected from ultraviolet light, visible light, and ionizing radiation.

10. (canceled)

11. (canceled)

12. A method of preventing radiation related skin damage in a subject through administering to the subject an effective amount of a composition comprising bixin or a variant thereof, wherein the radiation is UV-radiation, visible radiation, and/or ionizing radiation, wherein the composition comprising bixin further comprises polyethylene glycol.

13. The method of claim 12, wherein the effective amount of the composition is an amount capable of activating NRF2 pathway related activity in the subject, wherein said activating NRF2 pathway related activity in the subject occurs in keratinocyte cells and/or melanocyte cells.

14. (canceled)

15. The method of claim 12, wherein the effective amount of bixin within the composition is between 10 mg/kg and 200 mg/kg of the subject.

16. The method of claim 12, wherein the radiation related skin damage is one or more radiation related disorders selected from the group consisting of: advanced skin aging or wrinkling, thickening of the skin, uneven or pebbly skin, flabbiness, lifeless skin, pigmentation irregularities, small dilated blood vessels or red markings on or near the surface of the skin also known as telangiectasias, rough or scaly patches, e.g., actinic keratoses, freckles otherwise known as ephilides, liver spots, age spots, dark spots or skin tags known as lentigines, pre-skin cancers, and skin cancer, such as non-melanoma skin cancer (NMSC), e.g., superficial basal cell carcinoma (sBCC) and squamous cell carcinoma (SCC), and malignant melanoma.

17. The method of claim 12, wherein the composition is administered orally and/or topically.

18. (canceled)

19. (canceled)

20. The method of claim 12, wherein the amount of bixin within the composition comprising bixin and polyethylene glycol is approximately 1%.

21. The method of claim 12,
wherein the composition is co-administered with an additional composition comprising effective amounts of zinc oxide and/or titanium dioxide, or
wherein the composition further comprises effective amounts of zinc oxide and/or titanium oxide.

22. (canceled)

23. The method of claim 12, wherein the subject is a human subject.

24. The method of claim 12,
wherein the composition prevents pigment cells from pigment loss, and/or
wherein the composition prevents hair discoloration and/or hair aging, and/or
wherein the composition prevents vitiligo, and/or
wherein the composition prevents skin damage related to solar tanning.

25. (canceled)

26. (canceled)

27. (canceled)

28. The method of claim 12, wherein the bixin is irradiated, wherein the source of irradiation is selected from ultraviolet light, visible light, and ionizing radiation, wherein the result of irradiation is the formation of photochemical bixin derivatives and degradation products.

29. (canceled)

30. (canceled)

31. A kit comprising a composition comprising an effective amount of bixin or variant thereof, wherein the composition is capable of activating NRF2 pathway related activity, and instructions for administering the composition to a subject at risk for UV-radiation exposure, wherein the composition comprising an effective amount of bixin further comprises polyethylene glycol, wherein the amount of bixin within the composition comprising an effective amount of bixin and polyethylene glycol is approximately 1%, wherein the bixin is irradiated.

32. (canceled)

33. (canceled)

34. The kit of claim 31, wherein said activating NRF2 pathway related activity in the subject occurs in keratinocyte cells and/or melanocyte cells.

35. The kit of claim 31,
wherein the composition further comprises effective amounts of zinc oxide and/or titanium dioxide, and/or
wherein the kit further comprises an additional composition comprising effective amounts of zinc oxide and/or titanium dioxide.

36. (canceled)

37. (canceled)

38. The kit of claim 31, wherein the source of irradiation is selected from ultraviolet light, visible light, and ionizing radiation, wherein the result of irradiation is the formation of photochemical bixin derivatives and degradation products.

39-53. (canceled)


SEQUENCE LISTING (16 sequences)

SEQ ID NO: 1 - DNA, 20 bp, Mus musculus
ctcagcatgatggacttgga

SEQ ID NO: 2 - DNA, 20 bp, Mus musculus
tcttgcctccaaaggatgtc

SEQ ID NO: 3 - DNA, 19 bp, Mus musculus
gatcggctgcactgaactg

SEQ ID NO: 4 - DNA, 20 bp, Mus musculus
ggcagtgtgacaggttgaag

SEQ ID NO: 5 - DNA, 20 bp, Mus musculus
ggaggccatggagaagtgta

SEQ ID NO: 6 - DNA, 20 bp, Mus musculus
gcacacaggcttgtacctga

SEQ ID NO: 7 - DNA, 20 bp, Mus musculus
tcccatgcagtggagaagat

SEQ ID NO: 8 - DNA, 20 bp, Mus musculus
agctgtgcaactccaaggac

SEQ ID NO: 9 - DNA, 20 bp, Mus musculus
ccggagaggagacttcacag

SEQ ID NO: 10 - DNA, 20 bp, Mus musculus
tccacgatttcccagagaac

SEQ ID NO: 11 - DNA, 20 bp, Mus musculus
agcccccagtctgtatcctt

SEQ ID NO: 12 - DNA, 20 bp, Mus musculus
ggtcactgtcccagcatctt

SEQ ID NO: 13 - DNA, 20 bp, Mus musculus
caatccttgcaatgtggatg

SEQ ID NO: 14 - DNA, 20 bp, Mus musculus
agtaaggaaggggccctgta

SEQ ID NO: 15 - DNA, 20 bp, Mus musculus
aaggccaaccgtgaaaagat

SEQ ID NO: 16 - DNA, 21 bp, Mus musculus
gtggtacgaccagaggcatac